(19) United States
(12) Patent Application Publication
(10) Pub. No.: US 2017/0285010 A1
(43) Pub. Date: Oct. 5, 2017

(54) Analytical Methods and Arrays for Use in the Same

(71) Applicant: SENZAGEN AB, Lund (SE)

(72) Inventors: Malin Lindstedt, Sodra Sandby (SE); Carl A.K. Borrebaeck, Lund (SE); Henrik Johansson, Malmo (SE); Ann-Sofie Albrekt, Teckomatorp (SE); Andrew Forreryd, Malmo (SE)

(21) Appl. No.: 15/518,580

(22) PCT Filed: Nov. 27, 2015
(86) PCT No.: PCT/EP2015/077969

§ 371 Date: Apr. 12, 2017

(30) Foreign Application Priority Data
Nov. 28, 2014 (GB) 1421207.0

(51) Int. Cl. G01N 33/50; C12Q 1/68

(52) U.S. Cl. CPC G01N 33/5047; C12Q 1/6876; G01N 33/5023; C12Q 2600/158; G01N 2800/12; G01N 2333/70578; C12Q 2600/178; G01N 2333/924

(57) ABSTRACT

The present invention relates to a method for identifying agents capable of inducing respiratory sensitization in a mammal and arrays and diagnostic kits for use in such methods. In particular, the methods include measurement of the expression of the biomarkers listed in Table A(i), Table A(ii) and/or Table A(iii) in cells exposed to a test agent.

### FIELD OF THE INVENTION

The present invention relates to a method for identifying agents capable of inducing respiratory sensitization and arrays and analytical kits for use in such methods.

### BACKGROUND

Respiratory sensitization is an allergic type I hypersensitivity reaction of the upper and lower respiratory tract caused by an immune response towards environmental proteins or certain low molecular weight (LMW) chemical compounds. Clinical symptoms of respiratory sensitization, including wheezing, bronchoconstriction and asthmatic attacks, develops in susceptible and previously sensitized individuals upon repeated exposure to the same compound [1]. Mechanistically, respiratory sensitization is initiated by the activation of CD4+ Th2 cells and mediated by the differentiation of B-lymphocytes through the increased production of allergen specific IgE antibodies [1-3].

While respiratory allergy is generally induced by protein allergens, LMW chemical compounds have primarily been associated with the induction of type IV hypersensitivity reactions involving CD8+ T cells and CD4+ Th1 cells and the onset of skin conditions such as Allergic Contact Dermatitis (ACD). However, certain classes of LMW chemical compounds, such as diisocyanates [4], acid anhydrides [5], platinium salts [6], reactive dyes [7], and chloramine T [8] may also sensitize the respiratory tract. Exposure to these LMW chemical compounds is generally limited to occupational settings, and repeated exposure during an extended period of time may eventually result in development of occupational asthma (OA) [3,9]. Although fewer chemicals are known to cause respiratory allergy (<100 known substances) [10], compared to those causing contact dermatitis, health effects can still be disastrous. For example, acquired OA may result in chronic inflammation, airway hyperresponsiveness [11], extensive airway remodeling [12] and, thus, severely affecting the quality of life for affected individuals. The serious health effects associated with OA, as well as the introduction of new compounds into working environments (e.g cleaning agents and healthcare products [9,13-15]) highlights the need for accurate and reliable testing strategies for hazard classification of potential respiratory sensitizers. Proactive identification and characterization of these compounds thus remains an area of great importance.

A challenge in this respect, however, is that methods for risk assessment of chemicals inducing respiratory sensitization are greatly underdeveloped [16]. Current approaches involve both in-vivo and in-vitro testing strategies. Until recently, the field has relied on animal based in vivo models. Among these animal based approaches, guinea pig testing [17], mouse IgE testing [18,19], rat Ig E testing [20,21] and mouse cytokine fingerprinting [22,23] have gained most attention. In addition, several murine models of chemically induced asthma, aiming at discriminating respiratory sensitizers from skin sensitizers, are described in the literature [24,25]. Although these approaches undoubtedly have contributed to the current understanding of the immunobiological mechanisms and cellular processes associated with development of respiratory sensitization, none of the methods have proven sufficiently reliable in order to be used as a routine assay for regulatory purposes. Additionally, there are considerable economical and ethical drawbacks associated with the use of animal based methods as screening tools.

Considerable efforts have therefore been made to develop cell-based in-vitro assays for sensitization of the respiratory tract, which correlates with the principle of the three Rs on reduction, refinement and replacement of animal experiments stated in Directive 201/63/EU[26]. Recent cell-based approaches have involved the use of single cell lines as models for different stages in the sensitization process, such as the dendritic cell line THP-1[27] and the epithelial cell lines BEAS-2B[28] and A549[29]. More advanced attempts to mimic the in vivo route of exposure to respiratory sensitizers have also been performed, using the commercially available MucilAir™ developed by Epithelix as a 3D cell model of human airway epithelium [30]. In addition, non-cell based in silico predictive models based on chemical reactivity is being explored within respiratory sensitization [31].

In contrast to the lack of assays for respiratory sensitization, the literature describes several predictive models for identification of skin sensitizing chemicals (reviewed in [34]), with the animal based Local Lymph Node Assay (LLNA) [35] historically being the preferred method. Several in vitro models have also been described for the endpoint of skin sensitization, including the human Cell Line Activation Test (h-CLAT) [36,37], the Direct Peptide Activation Test (DRPA) [38] and the KeratinoSens® test [39,40]. Recently, we also presented our in-house developed Genomic Allergen Rapid Detection (GARD) [41,42] in vitro assay as an accurate alternative to these methods. The GARD assay is based on measurements of transcriptional levels of a genomic biomarker signature (GARD Prediction Signature, or GPS) comprising 200 genes in the myeloid human cell line MUTZ-3 [43-45] using transcriptome-wide DNA microarray technology. The functionality of the GARD assay in terms of predictive performance was evaluated in a recent study using a cohort comprising 26 blinded compounds and 11 non-blinded compounds. The accuracy of the assay was estimated to 89% [46], which can be compared to 72% for the LLNA [47]. Skin sensitization models are interesting because it has been proposed that skin sensitization assays, such as LLNA, could be applied to also classify respiratory sensitizers [48,49]. The endpoint in the LLNA assay is the provoked proliferative response measured in the draining lymph node upon topical exposure of mice to a test chemical [50,51]. However, this proliferative response is induced by both respiratory sensitizers and skin sensitizers. Consequently, while the LLNA can be used for stratification of sensitizers from non-sensitizers, it is unable to accurately discriminate between skin sensitizers and respiratory sensitizers [1].

Hence, there is a continuing need to establish accurate and reliable in vitro assays for specifically identifying respiratory sensitizers.

### DISCLOSURE OF THE INVENTION

The present invention concerns a cell based testing strategy for assessment of respiratory sensitizers based on a genomic biomarker signature as a novel alternative to animal testing. We utilized the great versatility that comes with analyzing the complete transcriptome of cells and extended the concept of the GARD assay to include prediction of respiratory sensitizers by identification of a separate genomic biomarker signature, called the GARD Respiratory Prediction Signature (GRPS). That can be used to classify respiratory sensitizers. The intended use of the identified biomarker signature will be in combination with GPS for classification of skin sensitizing chemicals. Thus, the GARD concept demonstrates a unique opportunity for a test platform that can simultaneously be used for risk assessment and hazard classification of both skin and respiratory sensitizing properties of unknown chemicals.

Hence, a first aspect of the present invention provides a method for identifying agents capable of inducing respiratory sensitization in a mammal comprising or consisting of the steps of:
a) exposing a population of dendritic cells or a population of dendritic-like cells to a test agent; and
b) measuring in the cells the expression of one or more biomarker(s) selected from the group defined in Table A,
   wherein the expression in the cells of the one or more biomarkers measured in step (b) is indicative of the respiratory sensitizing effect of the test agent.

By “indicative of the respiratory sensitizing effect of the test agent” we include determining whether or not the test agent is a respiratory sensitizer and/or determining the potency of the test agent as a respiratory sensitizer.

By “agents capable of inducing respiratory sensitization” we mean any agent capable of inducing and triggering a Type I immediate hypersensitivity reaction in the respiratory tract of a mammal. Preferably the mammal is a human. Preferably, the Type I immediate hypersensitivity reaction is DC-mediated and/or involves the differentiation of T cells into Th2 cells. Preferably the Type I immediate hypersensitivity reaction results in humoral immunity and/or respiratory allergy.

The conducting zone of the mammalian lung contains the trachea, the bronchi, the bronchioles, and the terminal bronchioles. The respiratory zone contains the respiratory bronchioles, the alveolar ducts, and the alveoli. The conducting zone is made up of airways, has no gas exchange with the blood, and is reinforced with cartilage in order to hold open the airways. The conducting zone humidifies inhaled air and warms it to 37° C. (99° F.). It also cleanses the air by removing particles via cilia located on the walls of all the passageways. The respiratory zone is the site of gas exchange with blood.

In one embodiment, the “agents capable of inducing sensitization of mammalian skin” is an agent capable of inducing and triggering a Type I immediate hypersensitivity reaction at a site of lung epithelium in a mammal. Preferably, the site of lung epithelium is in the respiratory zone of the lung, but may alternatively or additionally be in the conductive zone of the lung.

Preferably the method is an in vitro or ex vivo method.

The mammal may be any domestic or farm animal. Preferably, the mammal is a rat, mouse, guinea pig, cat, dog, horse or a primate. Most preferably, the mammal is human.

Dendritic cells (DCs) are immune cells forming part of the mammalian immune system. Their main function is to process antigen material and present it on the surface to other cells of the immune system (i.e., they function as antigen-presenting cells), bridging the innate and adaptive immune systems.

Dendritic cells are present in tissues in contact with the external environment, such as the skin (where there is a specialized dendritic cell type called Langerhans cells) and the inner lining of the nose, lungs, stomach and intestines. They can also be found in an immature state in the blood. Once activated, they migrate to the lymph nodes where they interact with T cells and B cells to initiate and shape the adaptive immune response. At certain development stages they grow branched projections, the dendrites. While similar in appearance, these are distinct structures from the dendrites of neurons. Immature dendritic cells are also called veiled cells, as they possess large cytoplasmic ‘veils’ rather than dendrites.

By “dendritic-like cells” we mean non-dendritic cells that exhibit functional and phenotypic characteristics specific to dendritic cells such as morphological characteristics, expression of costimulatory molecules and MHC class II molecules, and the ability to pinocytose macromolecules and to activate resting T cells.

In one embodiment, the dendritic-like cells are CD34⁺ dendritic cell progenitors. Optionally, the CD34⁺ dendritic cell progenitors can acquire, upon cytokine stimulation, the phenotypes of presenting antigens through CD1d, MHC class I and II, induce specific T-cell proliferation, and/or displaying a mature transcriptional and phenotypic profile upon stimulation with inflammatory mediators (i.e. similar phenotypes to immature dendritic cells or Langerhans-like dendritic cells).

Dendritic cells may be recognized by function, by phenotype and/or by gene expression pattern, particularly by cell surface phenotype. These cells are characterized by their distinctive morphology, high levels of surface MHC-class II expression and ability to present antigen to CD4+ and/or CD8+ T cells, particularly to naïve T cells (Steinman et al. (1991) Ann. Rev. Immunol. 9: 271).

The cell surface of dendritic cells is unusual, with characteristic veil-like projections, and is characterized by expression of the cell surface markers CD11c and MHC class II. Most DCs are negative for markers of other leukocyte lineages, including T cells, B cells, monocytes/macrophages, and granulocytes. Subpopulations of dendritic cells may also express additional markers selected from the group consisting of 33D1, CCR1, CCR2, CCR4, CCR5, CCR6, CCR7, CD1a-d, CD4, CD5, CD8alpha, CD9, CD11b, CD24, CD40, CD48, CD54, CD58, CD80, CD83, CD86, CD91, CD117, CD123 (IL3Ra), CD134, CD137, CD150, CD153, CD162, CXCR1, CXCR2, CXCR4, DCIR, DC-LAMP, DC-SIGN, DEC205, E-cadherin, Langerin, Mannose receptor, MARCO, TLR2, TLR3 TLR4, TLRS, TLR6, TLR9, CD14, CD34, HLA-DR and several lectins.

The patterns of expression of these cell surface markers may vary along with the maturity of the dendritic cells, their tissue of origin, and/or their species of origin. Immature dendritic cells express low levels of MHC class II, but are capable of endocytosing antigenic proteins and processing them for presentation in a complex with MHC class II molecules. Activated dendritic cells express high levels of MHC class 11, ICAM-1 and CD86, and are capable of stimulating the proliferation of naive allogeneic T cells, e. g. in a mixed leukocyte reaction (MLR).

Functionally, dendritic cells or dendritic-like cells may be identified by any convenient assay for determination of antigen presentation. Such assays may include testing the ability to stimulate antigen-primed and/or naive T cells by presentation of a test antigen, followed by determination of T cell proliferation, release of IL-2, and the like.

In one embodiment the dendritic-like cells include epithelial cells and/or epithelial-like cells such as BEAS-2B[28], WT 9-7 and A549[29]. Preferably the epithelial cells are lung epithelial cells. Preferably the epithelial-like cells are lung epithelial-like cells. In an alternative embodiment the dendritic-like cells include epithelial cells and/or epithelial-like cells.

By “expression” we mean the level or amount of a gene product such as mRNA or protein.

Methods of detecting and/or measuring the concentration of protein and/or nucleic acid are well known to those skilled in the art, see for example Sambrook and Russell, 2001, Cold Spring Harbor Laboratory Press.

Preferred methods for detection and/or measurement of protein include Western blot, North-Western blot, immunosorbent assays (ELISA), antibody microarray, tissue microarray (TMA), immunoprecipitation, in situ hybridisation and other immunohistochemistry techniques, radioimmunoassay (RIA), immunoradiometric assays (IRMA) and immunoenzymatic assays (IEMA), including sandwich assays using monoclonal and/or polyclonal antibodies. Exemplary sandwich assays are described by David et al., in U.S. Pat. Nos. 4,376,110 and 4,486,530, hereby incorporated by reference. Antibody staining of cells on slides may be used in methods well known in cytology laboratory diagnostic tests, as well known to those skilled in the art.

Typically, ELISA involves the use of enzymes which give a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemi-luminescent systems based on enzymes such as luciferase can also be used.

Conjugation with the vitamin biotin is frequently used since this can readily be detected by its reaction with enzyme-linked avidin or streptavidin to which it binds with great specificity and affinity.

Preferred methods for detection and/or measurement of nucleic acid (e.g. mRNA) include southern blot, northern blot, polymerase chain reaction (PCR), reverse transcriptase PCR (RT-PCR), quantitative real-time PCR (qRT-PCR), nanoarray, microarray, macroarray, autoradiography and in situ hybridisation.

In one embodiment the method further comprises the steps of:
c) exposing a separate population of the dendritic cells or dendritic-like cells to one or more negative control agent that is not a respiratory sensitizer in mammals; and
d) measuring in the cells the expression of the one or more biomarker(s) measured in step (b)
wherein the test agent is identified as a respiratory sensitizer in the event that the presence and/or amount in the test sample of the one or more biomarker measured in step (b) is different from the presence and/or amount in the control sample of the one or more biomarkers measured in step (d).

By “is different from the presence and/or amount in the control sample of the one or more proteins measured in step (b)” we mean that the presence and/or amount in the test sample differs from that of the one or more negative control sample in a statistically significant manner. Preferably the expression of the one or more biomarker in the cell population exposed to the test agent is:
less than or equal to 80% of that of the cell population exposed to the negative control agent, for example, no more than ₇₉%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1% or 0% of that of the cell population exposed to the negative control agent; or
at least 120% of that of the cell population exposed to the negative control agent, for example, at least 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 177%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 225%, 250%, 275%, 300%, 325%, 350%, 375%, 400%, 425%, 450%, 475% or at least 500% of that of the cell population exposed to the negative control agent

By “is different from the presence and/or amount in the control sample of the one or more proteins measured in step (b)” we alternatively or additionally include that the test sample is classified as belonging to a different group as the one or more negative control sample. For example, where an SVM is used, the test sample is on the other side of the decision value threshold as the one or more negative control sample (e.g., if the test agent is classified as a respiratory sensitizer if one or more test (or replicate thereof) has an SVM decision value of ≦0, then the one or more positive control samples (or the majority thereof) should also have an SVM decision value of ≦0).

The one or more negative control agent may comprise or consist of one or more agent selected from the group consisting of 1-Butanol; 2-Aminophenol; 2-Hydroxyethyl acrylate; 2-nitro-1,4-Phenylenediamine; 4-Aminobenzoic acid; Chlorobenzene; Dimethyl formamide; Ethyl vanillin; Formaldehyde; Geraniol; Hexylcinnamic aldehyde; Isopropanol; Kathon CG*; Methyl salicylate; Penicillin G; Propylene glycol; Potassium Dichromate; Potassium permanganate; Tween 80; and Zinc sulphate (i.e., the group of respiratory non-sensitizers defined in Table 1). The one or more negative control agent may comprise or consist of one or more agent selected from the group of negative control agents defined in Table 1.

The negative control agent may be a solvent for use with the test or control agents of the invention. Hence, the negative control may be DMSO and/or distilled water.

The method may comprise or consist of the use of at least 2 negative control agents (i.e. non-sensitizing agents), for example, at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or at least 100 negative control agents.

Alternatively or additionally, the expression of the one or more biomarkers measured in step (b) of the dendritic cells or dendritic-like cells prior to test agent exposure is used as a negative control.

A further embodiment comprises the steps of:
e) exposing a separate population of the dendritic cells or dendritic-like cells to one or more positive control agent that is a respiratory sensitizer in a mammal; and
f) measuring in the cells the expression of the one or more biomarker(s) measured in step (b)
   wherein the test agent is identified as a respiratory sensitizer in the event that the presence and/or amount in the test sample of the one or more biomarker measured in step (f) corresponds to the presence and/or amount in the one or more positive control sample of the one or more biomarker measured in step (b).

By “corresponds to the expression in the one or more positive control sample” we mean the expression of the one or more biomarker in the cell population exposed to the test agent is identical to, or does not differ significantly from, that of the cell population exposed to the one more positive control agent. Preferably the expression of the one or more biomarker in the cell population exposed to the test agent is between 81% and 119% of that of the cell population exposed to the one more positive control agent, for example, greater than or equal to 82%, 83%, 84%, 85%, 86%, 87%, 88%, 89%, 90%, 91%, 92%, 93%, 94%, 95%, 96%, 97%, 98% or 99% of that of the cell population exposed to the one more positive control agent, and less than or equal to 101%, 102%, 103%, 104%, 105%, 106%, 107%, 108%, 109%, 110%, 111%, 112%, 113%, 114%, 115%, 116%, 117%, 118% or 119% of that of the cell population exposed to the one more positive control agent.

By “corresponds to the expression in the one or more positive control sample” we alternatively or additionally include that the test sample is classified as belonging to the same group as the one or more positive control sample. For example, where an SVM is used, the test sample is on the same side of the decision value threshold as the one or more positive control sample (e.g., if the test agent is classified as a respiratory sensitizer if one or more test (or replicate thereof) has an SVM decision value of >0, then the one or more positive control samples (or the majority thereof) should also have an SVM decision value of >0).

The one or more positive control agent may comprise or consist of one or more agent selected from the group consisting of Ammonium hexachloroplatinate; Ammonium persulfate; Ethylenediamine; Glutaraldehyde; Hexamethylen diisocyanate; Maleic Anhydride; Methylene diphenol diisocyanate; Phtalic Anhydride; Toluendiisocyanate; and Trimellitic anhydride (i.e., the group of respiratory sensitizers defined in Table 1).

The one or more positive control agent may comprise or consist of one or more agent selected from the group of positive control agents defined in Table 1.

The method may comprise or consist of the use of at least 2 positive control (i.e. sensitizing agents), for example, at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or at least 100 positive control agents.

The method according to the first aspect of the invention may include or exclude measuring the expression of TNFRSF19. The method may include or exclude measuring the expression of SNORA74A. The method may include or exclude measuring the expression of SPAM1.

The method may include or exclude measuring the expression of Ensembl transcript identification number (ETID): ENST00000364621; The method may include or exclude measuring the expression of HOMER3; The method may include or exclude measuring the expression of CD1C; The method may include or exclude measuring the expression of IGHD///IGHM; The method may include or exclude measuring the expression of SNRPN///SNORD116-26; The method may include or exclude measuring the expression of ETID: ENST00000364678; The method may include or exclude measuring the expression of STRAP; The method may include or exclude measuring the expression of DIABLO; The method may include or exclude measuring the expression of ETID: ENST00000411349; The method may include or exclude measuring the expression of ETID: ENST00000385497; The method may include or exclude measuring the expression of OR51A2; The method may include or exclude measuring the expression of MRPL21; The method may include or exclude measuring the expression of PPP1R14A; The method may include or exclude measuring the expression of DEFB127; The method may include or exclude measuring the expression of C9orf130; The method may include or exclude measuring the expression of PRO2012; The method may include or exclude measuring the expression of LOC399898; The method may include or exclude measuring the expression of ETID: ENST00000387701; The method may include or exclude measuring the expression of WDR68; The method may include or exclude measuring the expression of NEU2; The method may include or exclude measuring the expression of ETID: ENST00000386677; The method may include or exclude measuring the expression of SPARC; The method may include or exclude measuring the expression of ETID: ENST00000390342; The method may include or exclude measuring the expression of CRNN; The method may include or exclude measuring the expression of MMP12; The method may include or exclude measuring the expression of ACVRL1; The method may include or exclude measuring the expression of EIF4E2; The method may include or exclude measuring the expression of RP11-191L9.1; The method may include or exclude measuring the expression of PDCD6///AHRR; The method may include or exclude measuring the expression of ARRDC3; The method may include or exclude measuring the expression of VWDE;

The method may include or exclude measuring the expression of ZBTB34; The method may include or exclude measuring the expression of ITGB1 BP2; The method may include or exclude measuring the expression of OR10K2; The method may include or exclude measuring the expression of FLJ22596; The method may include or exclude measuring the expression of ETID: ENST00000306515; The method may include or exclude measuring the expression of ACVR2A; The method may include or exclude measuring the expression of ETID: ENST00000385690; The method may include or exclude measuring the expression of ETID: ENST00000386018; The method may include or exclude measuring the expression of C6orf201; The method may include or exclude measuring the expression of ETID: ENST00000385583; The method may include or exclude measuring the expression of ETID: ENST00000385719; The method may include or exclude measuring the expression of GPR20; The method may include or exclude measuring the expression of ETID: ENST00000364357; The method may include or exclude measuring the expression of ZCCHC13; The method may include or exclude measuring the expression of GPR64; The method may include or exclude measuring the expression of CD1D; The method may include or exclude measuring the expression of DUSP12; The method may include or exclude measuring the expression of KLHL33; The method may include or exclude measuring the expression of PSMB6; The method may include or exclude measuring the expression of TMEM95; The method may include or exclude measuring the expression of C1QBP; The method may include or exclude measuring the expression of EMILIN2; The method may include or exclude measuring the expression of CD8A; The method may include or exclude measuring the expression of C20orf152; The method may include or exclude measuring the expression of KCNJ4; The method may include or exclude measuring the expression of ETID: ENST00000364163; The method may include or exclude measuring the expression of FAM19A1; The method may include or exclude measuring the expression of ETID: ENST00000384601; The method may include or exclude measuring the expression of POLR2H; The method may include or exclude measuring the expression of AK000420; The method may include or exclude measuring the expression of ETID: ENST00000363354; The method may include or exclude measuring the expression of Affymetrix probe set identification number (APID): 8121483; The method may include or exclude measuring the expression of EGFL6; The method may include or exclude measuring the expression of POU3F4; The method may include or exclude measuring the expression of ETID: ENST00000385841; The method may include or exclude measuring the expression of OR52A5; The method may include or exclude measuring the expression of TIMM8B; The method may include or exclude measuring the expression of PEBP1; The method may include or exclude measuring the expression of OR4F6; The method may include or exclude measuring the expression of CDH15; The method may include or exclude measuring the expression of TMEM199; The method may include or exclude measuring the expression of AB13; The method may include or exclude measuring the expression of FLJ42842; The method may include or exclude measuring the expression of MC4R; The method may include or exclude measuring the expression of ETID: ENST00000410673; The method may include or exclude measuring the expression of ISM1; The method may include or exclude measuring the expression of LOC440957; The method may include or exclude measuring the expression of KLB; The method may include or exclude measuring the expression of GM2A; The method may include or exclude measuring the expression of ANXA6; The method may include or exclude measuring the expression of TAS2R40; The method may include or exclude measuring the expression of APID: 8142880; The method may include or exclude measuring the expression of RARRES2; The method may include or exclude measuring the expression of SH2D4A; The method may include or exclude measuring the expression of PLP1; The method may include or exclude measuring the expression of ATP1A2; The method may include or exclude measuring the expression of ETID: ENST00000386800; The method may include or exclude measuring the expression of MAT1A; The method may include or exclude measuring the expression of TSGA10IP; The method may include or exclude measuring the expression of PRDM7; The method may include or exclude measuring the expression of ETID: ENST00000390847; The method may include or exclude measuring the expression of ETID: ENST00000255183; The method may include or exclude measuring the expression of MRPL39; The method may include or exclude measuring the expression of ETID: ENST00000386327; The method may include or exclude measuring the expression of TIPARP; The method may include or exclude measuring the expression of HES1; The method may include or exclude measuring the expression of ETID: ENST00000363502; The method may include or exclude measuring the expression of PRDM9; The method may include or exclude measuring the expression of ETID: ENST00000390917; The method may include or exclude measuring the expression of KIAA1688; The method may include or exclude measuring the expression of ETID: ENST00000391219; The method may include or exclude measuring the expression of ETID: ENST00000387973; The method may include or exclude measuring the expression of LOC100129534; The method may include or exclude measuring the expression of SLC2A1; The method may include or exclude measuring the expression of AF116714; The method may include or exclude measuring the expression of EPS8L2; The method may include or exclude measuring the expression of MGC3196; The method may include or exclude measuring the expression of 7952733; The method may include or exclude measuring the expression of ETID: ENST00000384391; The method may include or exclude measuring the expression of EME2; The method may include or exclude measuring the expression of NETO1; The method may include or exclude measuring the expression of NPHS1; The method may include or exclude measuring the expression of ETID: ENST00000384109; The method may include or exclude measuring the expression of ETID: ENST00000364143; The method may include or exclude measuring the expression of ISX; The method may include or exclude measuring the expression of IL17RB; The method may include or exclude measuring the expression of PCOLCE2; The method may include or exclude measuring the expression of LRIT3; The method may include or exclude measuring the expression of ETID: ENST00000330110; The method may include or exclude measuring the expression of ZNF354C; The method may include or exclude measuring the expression of ETID: ENST00000386444; The method may include or exclude measuring the expression of OR2G3; The method may include or exclude measuring the expression of GLUL; The method may include or exclude measuring the expression of CCKBR; The method may include or exclude measuring the expression of OR1S2; The method may include or exclude measuring the expression of DCUN1D5; The method may include or exclude measuring the expression of ETID: ENST00000388291; The method may include or exclude measuring the expression of EMG1; The method may include or exclude measuring the expression of PTHLH; The method may include or exclude measuring the expression of PTGES3; The method may include or exclude measuring the expression of CIDEB; The method may include or exclude measuring the expression of ETID: ENST00000383863; The method may include or exclude measuring the expression of ATP10A; The method may include or exclude measuring the expression of MYO5C; The method may include or exclude measuring the expression of ETID: ENST00000380078; The method may include or exclude measuring the expression of PLA2G10; The method may include or exclude measuring the expression of HSPE1; The method may include or exclude measuring the expression of ETID: ENST00000388324; The method may include or exclude measuring the expression of MYO6; The method may include or exclude measuring the expression of C7orf30; The method may include or exclude measuring the expression of ETID: ENST00000340779; The method may include or exclude measuring the expression of LOC441245; The method may include or exclude measuring the expression of CRIM2; The method may include or exclude measuring the expression of XKR4; The method may include or exclude measuring the expression of FAM110B; The method may include or exclude measuring the expression of PEBP4; The method may include or exclude measuring the expression of LOC644714; The method may include or exclude measuring the expression of PAPPAS; The method may include or exclude measuring the expression of BEX4; The method may include or exclude measuring the expression of HMGB4; The method may include or exclude measuring the expression of ETID: BCO28413///BC128516; The method may include or exclude measuring the expression of ETID: ENST00000363919; The method may include or exclude measuring the expression of ETID: ENST00000335621; The method may include or exclude measuring the expression of SOX1; The method may include or exclude measuring the expression of CTSG; The method may include or exclude measuring the expression of ETID: ENST00000362344; The method may include or exclude measuring the expression of FLJ37464; The method may include or exclude measuring the expression of RAX; The method may include or exclude measuring the expression of IL29; The method may include or exclude measuring the expression of CEACAM20; The method may include or exclude measuring the expression of ETID: ETID: ENST00000365557; The method may include or exclude measuring the expression of SEC14L3; The method may include or exclude measuring the expression of C3orf52; The method may include or exclude measuring the expression of FETUB; The method may include or exclude measuring the expression of PIGY; The method may include or exclude measuring the expression of CDH12; The method may include or exclude measuring the expression of LGSN; The method may include or exclude measuring the expression of ETID: ENST00000391031; The method may include or exclude measuring the expression of HGC6.3; The method may include or exclude measuring the expression of tcag7.873; The method may include or exclude measuring the expression of T1560;

The method may include or exclude measuring the expression of EXOSC4; The method may include or exclude measuring the expression of TRAM1; The method may include or exclude measuring the expression of APID: 8159371; The method may include or exclude measuring the expression of OR13C2; The method may include or exclude measuring the expression of PLS3; The method may include or exclude measuring the expression of TMEM53; The method may include or exclude measuring the expression of CD1B; The method may include or exclude measuring the expression of SORCS3; The method may include or exclude measuring the expression of OR52E8; The method may include or exclude measuring the expression of FAM160A2; The method may include or exclude measuring the expression of LOC649946; The method may include or exclude measuring the expression of FAM158A; The method may include or exclude measuring the expression of APID: 7986637; The method may include or exclude measuring the expression of MYO1E; The method may include or exclude measuring the expression of NUPR1; The method may include or exclude measuring the expression of APID: 8005433; The method may include or exclude measuring the expression of SIGLEC15; The method may include or exclude measuring the expression of 2-Mar; The method may include or exclude measuring the expression of LOC100131554; The method may include or exclude measuring the expression of GGTLC1; The method may include or exclude measuring the expression of PSMA7; The method may include or exclude measuring the expression of SLC25A18; The method may include or exclude measuring the expression of C3orf14; The method may include or exclude measuring the expression of CDX1; The method may include or exclude measuring the expression of ETID: ENST00000386433; The method may include or exclude measuring the expression of RRAGD; The method may include or exclude measuring the expression of SDK1; The method may include or exclude measuring the expression of LOC168474; The method may include or exclude measuring the expression of ETID: ENST00000384125; The method may include or exclude measuring the expression of TRHR; The method may include or exclude measuring the expression of 11_11RA; The method may include or exclude measuring the expression of MGC21881///L00554249; The method may include or exclude measuring the expression of ZNF483; The method may include or exclude measuring the expression of C9orf169; The method may include or exclude measuring the expression of MGC21881///L00554249; The method may include or exclude measuring the expression of ETID: ENST00000364507; The method may include or exclude measuring the expression of ETID: ENST00000387003; The method may include or exclude measuring the expression of ETID: ENST00000388083; The method may include or exclude measuring the expression of ETID: ENST00000365084; The method may include or exclude measuring the expression of FRG2///FRG2B /// FRG2C; The method may include or exclude measuring the expression of C14orf53; The method may include or exclude measuring the expression of ODF3L1; The method may include or exclude measuring the expression of FAM18A; The method may include or exclude measuring the expression of PRTN3; The method may include or exclude measuring the expression of CFD; The method may include or exclude measuring the expression of TMED1; The method may include or exclude measuring the expression of ETID: ENST00000387150; The method may include or exclude measuring the expression of HSD17B14; The method may include or exclude measuring the expression of BOK; The method may include or exclude measuring the expression of ETID: ENST00000365609; The method may include or exclude measuring the expression of SNRPB; The method may include or exclude measuring the expression of EPHA6; The method may include or exclude measuring the expression of SCARNA22; The method may include or exclude measuring the expression of FLJ35424; The method may include or exclude measuring the expression of ETID: ENST00000387555; The method may include or exclude measuring the expression of ETID: ENST00000388664; The method may include or exclude measuring the expression of ETID: ENST00000363365; The method may include or exclude measuring the expression of ETID: ENST00000362861; The method may include or exclude measuring the expression of ETID: ENST00000363181; The method may include or exclude measuring the expression of GRM6; The method may include or exclude measuring the expression of LOC646093; The method may include or exclude measuring the expression of HIST1H1E; The method may include or exclude measuring the expression of TIAM2; The method may include or exclude measuring the expression of ETID: ENST00000363074; The method may include or exclude measuring the expression of ETID: ENST00000385777; The method may include or exclude measuring the expression of MTUS1; The method may include or exclude measuring the expression of MUC21; The method may include or exclude measuring the expression of WDR8; The method may include or exclude measuring the expression of LOC100131195; The method may include or exclude measuring the expression of OR4D10; The method may include or exclude measuring the expression of C12orf63; The method may include or exclude measuring the expression of ELA1; The method may include or exclude measuring the expression of DNAJC14///CIP29; The method may include or exclude measuring the expression of FLJ40176; The method may include or exclude measuring the expression of ETID: ENST00000410207; The method may include or exclude measuring the expression of PSME3; The method may include or exclude measuring the expression of ETID: ENST00000405656; The method may include or exclude measuring the expression of HN1; The method may include or exclude measuring the expression of ETID: ENST00000335523; The method may include or exclude measuring the expression of CYP2A7///CYP2A7P1; The method may include or exclude measuring the expression of ATXN10; The method may include or exclude measuring the expression of ZMATS; The method may include or exclude measuring the expression of ETID: ENST00000362493; The method may include or exclude measuring the expression of FHIT; The method may include or exclude measuring the expression of FRG2///FRG2B /// FRG2C; The method may include or exclude measuring the expression of SNX18; The method may include or exclude measuring the expression of ETID: ENST00000362433; The method may include or exclude measuring the expression of DTX2; The method may include or exclude measuring the expression of ASB4; The method may include or exclude measuring the expression of ETID: ENST00000365242; The method may include or exclude measuring the expression of ETID: ENST00000364204; The method may include or exclude measuring the expression of COL5A1; The method may include or exclude measuring the expression of LCAP; The method may include or exclude measuring the expression of APOO; The method may include or exclude measuring the expression of PTPRU; The method may include or exclude measuring the expression of IL28RA; The method may include or exclude measuring the expression of NEUROG3; The method may include or exclude measuring the expression of VAX1; The method may include or exclude measuring the expression of LOC440131; The method may include or exclude measuring the expression of C13orf31; The method may include or exclude measuring the expression of ADAMTS7; The method may include or exclude measuring the expression of SMTNL2; The method may include or exclude measuring the expression of LOC284112; The method may include or exclude measuring the expression of ETV2; The method may include or exclude measuring the expression of FUT2; The method may include or exclude measuring the expression of C2orf39; The method may include or exclude measuring the expression of LOC200383///DNAH6; The method may include or exclude measuring the expression of ETID: ENST00000385676; The method may include or exclude measuring the expression of CCDC108; The method may include or exclude measuring the expression of APID: 8065011; The method may include or exclude measuring the expression of C22orf27; The method may include or exclude measuring the expression of ETID: ENST00000364444; The method may include or exclude measuring the expression of PDLIM3; The method may include or exclude measuring the expression of ETID: ENST00000330110; The method may include or exclude measuring the expression of ETID: ENST00000384539; The method may include or exclude measuring the expression of ETID: ENST00000390214; The method may include or exclude measuring the expression of MGC72080; The method may include or exclude measuring the expression of C9orf128; The method may include or exclude measuring the expression of RGAG4; The method may include or exclude measuring the expression of PIP5K1A; The method may include or exclude measuring the expression of GPR161; The method may include or exclude measuring the expression of ETID: ENST00000385353; The method may include or exclude measuring the expression of OR56A3; The method may include or exclude measuring the expression of OR5A2; The method may include or exclude measuring the expression of WNT11; The method may include or exclude measuring the expression of APID: 7960259; The method may include or exclude measuring the expression of RAB37; The method may include or exclude measuring the expression of LAIR1; The method may include or exclude measuring the expression of ETID: ENST00000388385; The method may include or exclude measuring the expression of CHAC2; The method may include or exclude measuring the expression of ETID: ENST00000387574; The method may include or exclude measuring the expression of ETID: ENST00000387884; The method may include or exclude measuring the expression of BCL2L1; The method may include or exclude measuring the expression of KDELR3; The method may include or exclude measuring the expression of TMEM108; The method may include or exclude measuring the expression of SPATA16; The method may include or exclude measuring the expression of BTC; The method may include or exclude measuring the expression of SUPT3H; The method may include or exclude measuring the expression of EIF4B; The method may include or exclude measuring the expression of CHMP4C; The method may include or exclude measuring the expression of H2BFM; The method may include or exclude measuring the expression of APID: 8180392; The method may include or exclude measuring the expression of NR5A2; The method may include or exclude measuring the expression of TRIM49; The method may include or exclude measuring the expression of MS4A6A; The method may include or exclude measuring the expression of C11orf10; The method may include or exclude measuring the expression of HSPC152; The method may include or exclude measuring the expression of RASAL1; The method may include or exclude measuring the expression of ETID: ENST00000387531; The method may include or exclude measuring the expression of PLDN; The method may include or exclude measuring the expression of PERI; The method may include or exclude measuring the expression of ALS2CR12; The method may include or exclude measuring the expression of C20orf142; The method may include or exclude measuring the expression of ETID: ENST00000386848; The method may include or exclude measuring the expression of LOC100129113; The method may include or exclude measuring the expression of CERK; The method may include or exclude measuring the expression of ETID: ENST00000385783; The method may include or exclude measuring the expression of PROS1; The method may include or exclude measuring the expression of PCDHGA; The method may include or exclude measuring the expression of MUC3B /// MUC3A; The method may include or exclude measuring the expression of ETID: ENST00000365355; The method may include or exclude measuring the expression of APID: 8156969; The method may include or exclude measuring the expression of ETID: ENST00000358047; The method may include or exclude measuring the expression of FAM47C; The method may include or exclude measuring the expression of NXF4; The method may include or exclude measuring the expression of PIWIL4; The method may include or exclude measuring the expression of ETID: ENST00000384727; The method may include or exclude measuring the expression of ALDH6A1; The method may include or exclude measuring the expression of TMEM64; The method may include or exclude measuring the expression of ETID: ENST00000364816.

The method may include or exclude measuring the expression of C11orf73; The method may include or exclude measuring the expression of OR5B21; The method may include or exclude measuring the expression of NOXS///SPESP1; The method may include or exclude measuring the expression of AMICA1; The method may include or exclude measuring the expression of ETID: ENST00000387422; The method may include or exclude measuring the expression of SERPINB1; The method may include or exclude measuring the expression of ETID: ENST00000387396; The method may include or exclude measuring the expression of CD1A; The method may include or exclude measuring the expression of RAB9A; The method may include or exclude measuring the expression of C10orf90; The method may include or exclude measuring the expression of LPXN; The method may include or exclude measuring the expression of GGTLC2; The method may include or exclude measuring the expression of ETID: ENST00000384680; The method may include or exclude measuring the expression of PNPLA4; The method may include or exclude measuring the expression of CAMK1D; The method may include or exclude measuring the expression of ETID: ENST00000410754; The method may include or exclude measuring the expression of CDC123; The method may include or exclude measuring the expression of WDFY1; The method may include or exclude measuring the expression of hCG_1749005; The method may include or exclude measuring the expression of CD48; The method may include or exclude measuring the expression of MED19; The method may include or exclude measuring the expression of DRD5; The method may include or exclude measuring the expression of APID: 7967586; The method may include or exclude measuring the expression of VAPA; The method may include or exclude measuring the expression of FAM71F1; The method may include or exclude measuring the expression of APID: 8141421; The method may include or exclude measuring the expression of HCCS; The method may include or exclude measuring the expression of CNR2; The method may include or exclude measuring the expression of OIT3; The method may include or exclude measuring the expression of BMP2K; The method may include or exclude measuring the expression of ZNF366; The method may include or exclude measuring the expression of SYT17; The method may include or exclude measuring the expression of CALM2; The method may include or exclude measuring the expression of XK; The method may include or exclude measuring the expression of ART4; The method may include or exclude measuring the expression of ETID: ENST00000332418; The method may include or exclude measuring the expression of ZFP36L2; The method may include or exclude measuring the expression of GSTA3; The method may include or exclude measuring the expression of COL21A1; The method may include or exclude measuring the expression of ETID: ENST00000332418; The method may include or exclude measuring the expression of FUCA1; The method may include or exclude measuring the expression of ETID: ENST00000386628; The method may include or exclude measuring the expression of AZU1; The method may include or exclude measuring the expression of IL7R.

The method may comprise or consist of measuring, in step (b), the expression of one or more biomarkers defined in Table A(i), for example, at least 2 or 3 of the biomarkers defined in Table 1A. Hence, the method may comprise measuring the expression of TNFRSF19. The method may comprise measuring the expression of SNORA74A. The method may comprise measuring the expression of SPAM1. In a preferred embodiment, the method comprises or consists of measuring the expression of TNFRSF19, SNORA74 Aand SPAM1in step (b).

The method may additionally or alternatively comprise or consist of, measuring in step (b) the expression of one or more biomarkers defined in Table A(ii), for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341 or 342 of the biomarkers defined in Table A(ii).

The method may additionally or alternatively comprise or consist of, measuring in step (b) the expression of one or more biomarkers defined in Table 10, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43 or 44 of the biomarkers defined in Table A(iii).

Thus, the expression of all of the biomarkers defined in Table A(i) and/or all of the biomarkers defined in Table A(ii) and/or all of the biomarkers defined in Table A(iii) may be measured in step (b). Hence, the method may comprise or consist of measuring in step (b) all of the biomarkers defined in Table A.

In a preferred embodiment, step (b) comprises or consists of measuring the expression of a nucleic acid molecule encoding the one or more biomarker(s). The nucleic acid molecule may be a cDNA molecule or an mRNA molecule. Preferably, the nucleic acid molecule is an mRNA molecule. However, the nucleic acid molecule may be a cDNA molecule.

In one embodiment the expression of the one or more biomarker(s) in step (b) is performed using a method selected from the group consisting of Southern hybridisation, Northern hybridisation, polymerase chain reaction (PCR), reverse transcriptase PCR (RT-PCR), quantitative real-time PCR (qRT-PCR), nanoarray, microarray, macroarray, autoradiography and in situ hybridisation. Preferably, the expression of the one or more biomarker(s) is measured using a DNA microarray.

The method may comprise measuring the expression of the one or more biomarker(s) in step (b) using one or more binding moieties, each capable of binding selectively to a nucleic acid molecule encoding one of the biomarkers identified in Table A.

In one embodiment the one or more binding moieties each comprise or consist of a nucleic acid molecule. In a further embodiment the one or more binding moieties each comprise or consist of DNA, RNA, PNA, LNA, GNA, TNA or PMO. Preferably, the one or more binding moieties each comprise or consist of DNA. In one embodiment, the one or more binding moieties are 5 to 100 nucleotides in length. However, in an alternative embodiment, they are 15 to 35 nucleotides in length.

The one or more binding moieties may comprise or consist of one or more probe from the Human Gene 1.0 ST Array (Affymetrix, Santa Clara, Calif., USA). Probe identification numbers are provided in Table A herein.

Suitable binding agents (also referred to as binding molecules) may be selected or screened from a library based on their ability to bind a given nucleic acid, protein or amino acid motif, as discussed below.

In a preferred embodiment, the binding moiety comprises a detectable moiety.

By a “detectable moiety” we include a moiety which permits its presence and/or relative amount and/or location (for example, the location on an array) to be determined, either directly or indirectly.

Suitable detectable moieties are well known in the art.

For example, the detectable moiety may be a fluorescent and/or luminescent and/or chemiluminescent moiety which, when exposed to specific conditions, may be detected. Such a fluorescent moiety may need to be exposed to radiation (i.e. light) at a specific wavelength and intensity to cause excitation of the fluorescent moiety, thereby enabling it to emit detectable fluorescence at a specific wavelength that may be detected.

Alternatively, the detectable moiety may be an enzyme which is capable of converting a (preferably undetectable) substrate into a detectable product that can be visualised and/or detected. Examples of suitable enzymes are discussed in more detail below in relation to, for example, ELISA assays.

Hence, the detectable moiety may be selected from the group consisting of: a fluorescent moiety; a luminescent moiety; a chemiluminescent moiety; a radioactive moiety (for example, a radioactive atom); or an enzymatic moiety. Preferably, the detectable moiety comprises or consists of a radioactive atom. The radioactive atom may be selected from the group consisting of technetium-99m, iodine-123, iodine-125, iodine-131, indium-111, fluorine-19, carbon-13, nitrogen-15, oxygen-17, phosphorus-32, sulphur-35, deuterium, tritium, rhenium-186, rhenium-188 and yttrium-90.

Clearly, the agent to be detected (such as, for example, the one or more biomarkers in the test sample and/or control sample described herein and/or an antibody molecule for use in detecting a selected protein) must have sufficient of the appropriate atomic isotopes in order for the detectable moiety to be readily detectable.

In an alternative preferred embodiment, the detectable moiety of the binding moiety is a fluorescent moiety.

The radio- or other labels may be incorporated into the biomarkers present in the samples of the methods of the invention and/or the binding moieties of the invention in known ways. For example, if the binding agent is a polypeptide it may be biosynthesised or may be synthesised by chemical amino acid synthesis using suitable amino acid precursors involving, for example, fluorine-19 in place of hydrogen. Labels such as ^{99m}Tc, ¹²³, ¹⁸⁶Rh, ¹⁸⁸Rh and ¹¹¹In can, for example, be attached via cysteine residues in the binding moiety. Yttrium-90 can be attached via a lysine residue. The IODOGEN method (Fraker et al (1978) Biochem. Biophys. Res. Comm. 80, 49-57) can be used to incorporate ¹²³I. Reference (“Monoclonal Antibodies in Immunoscintigraphy”, J-F Chatal, CRC Press, 1989) describes other methods in detail. Methods for conjugating other detectable moieties (such as enzymatic, fluorescent, luminescent, chemiluminescent or radioactive moieties) to proteins are well known in the art.

It will be appreciated by persons skilled in the art that biomarkers in the sample(s) to be tested may be labelled with a moiety which indirectly assists with determining the presence, amount and/or location of said proteins. Thus, the moiety may constitute one component of a multicomponent detectable moiety. For example, the biomarkers in the sample(s) to be tested may be labelled with biotin, which allows their subsequent detection using streptavidin fused or otherwise joined to a detectable label.

The method provided in the first aspect of the present invention may comprise or consist of, in step (b), determining the expression of the protein of the one or more biomarker defined in Table A. The method may comprise measuring the expression of the one or more biomarker(s) in step (b) using one or more binding moieties each capable of binding selectively to one of the biomarkers identified in Table A. The one or more binding moieties may comprise or consist of an antibody or an antigen-binding fragment thereof such as a monoclonal antibody or fragment thereof.

The term “antibody” includes any synthetic antibodies, recombinant antibodies or antibody hybrids, such as but not limited to, a single-chain antibody molecule produced by phage-display of immunoglobulin light and/or heavy chain variable and/or constant regions, or other immunointeractive molecules capable of binding to an antigen in an immunoassay format that is known to those skilled in the art.

We also include the use of antibody-like binding agents, such as affibodies and aptamers.

A general review of the techniques involved in the synthesis of antibody fragments which retain their specific binding sites is to be found in Winter & Milstein (1991) Nature 349, 293-299.

Additionally, or alternatively, one or more of the first binding molecules may be an aptamer (see Collett et al., 2005, Methods 37:4-15).

Molecular libraries such as antibody libraries (Clackson et al, 1991, Nature 352, 624-628; Marks et al, 1991, J Mol Biol 222(3): 581-97), peptide libraries (Smith, 1985, Science 228(4705): 1315-7), expressed cDNA libraries (Santi et al (2000) J Mol Biol 296(2): 497-508), libraries on other scaffolds than the antibody framework such as affibodies (Gunneriusson et al, 1999, Appl Environ Microbiol 65(9): 4134-40) or libraries based on aptamers (Kenan et al, 1999, Methods Mol Biol 118, 217-31) may be used as a source from which binding molecules that are specific for a given motif are selected for use in the methods of the invention.

The molecular libraries may be expressed in vivo in prokaryotic cells (Clackson et al, 1991, op. cit.; Marks et al, 1991, op. cit.) or eukaryotic cells (Kieke et al, 1999, Proc Natl Acad Sci USA, 96(10):5651-6) or may be expressed in vitro without involvement of cells (Hanes & Pluckthun, 1997, Proc Natl Acad Sci USA 94(10):4937-42; He & Taussig, 1997, Nucleic Acids Res 25(24):5132-4; Nemoto et al, 1997, FEBS Lett, 414(2):405-8).

In cases when protein based libraries are used, the genes encoding the libraries of potential binding molecules are often packaged in viruses and the potential binding molecule displayed at the surface of the virus (Clackson et al, 1991, supra; Marks et al, 1991, supra; Smith, 1985, supra).

Perhaps the most commonly used display system is filamentous bacteriophage displaying antibody fragments at their surfaces, the antibody fragments being expressed as a fusion to the minor coat protein of the bacteriophage (Clackson et al, 1991, supra; Marks et al, 1991, supra). However, other suitable systems for display include using other viruses (EP 39578), bacteria (Gunneriusson et al, 1999, supra; Daugherty et al, 1998, Protein Eng 11(9):825-32; Daugherty et al, 1999, Protein Eng 12(7):613-21), and yeast (Shusta et al, 1999, J Mol Biol 292(5):949-56).

In addition, display systems have been developed utilising linkage of the polypeptide product to its encoding mRNA in so-called ribosome display systems (Hanes &

Pluckthun, 1997, supra; He & Taussig, 1997, supra; Nemoto et al, 1997, supra), or alternatively linkage of the polypeptide product to the encoding DNA (see U.S. Pat. No. 5,856,090 and WO 98/37186).

The variable heavy (VH) and variable light (VL) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sci. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

The antibody or antigen-binding fragment may be selected from the group consisting of intact antibodies, Fv fragments (e.g. single chain Fv and disulphide-bonded Fv), Fab-like fragments (e.g. Fab fragments, Fab′ fragments and F(ab)₂ fragments), single variable domains (e.g. V_{H} and V_{L} domains) and domain antibodies (dAbs, including single and dual formats [i.e. dAb-linker-dAb]). Preferably, the antibody or antigen-binding fragment is a single chain Fv (scFv).

The one or more binding moieties may alternatively comprise or consist of an antibody-like binding agent, for example an affibody or aptamer.

By “scFv molecules” we mean molecules wherein the VH and VL partner domains are linked via a flexible oligopeptide.

The advantages of using antibody fragments, rather than whole antibodies, are several-fold. The smaller size of the fragments may lead to improved pharmacological properties, such as better penetration of solid tissue. Effector functions of whole antibodies, such as complement binding, are removed. Fab, Fv, ScFv and dAb antibody fragments can all be expressed in and secreted from E. coli, thus allowing the facile production of large amounts of the said fragments.

Whole antibodies, and F(ab′)2 fragments are “bivalent”. By “bivalent” we mean that the said antibodies and F(ab′)2 fragments have two antigen combining sites. In contrast, Fab, Fv, ScFv and dAb fragments are monovalent, having only one antigen combining sites.

The antibodies may be monoclonal or polyclonal. Suitable monoclonal antibodies may be prepared by known techniques, for example those disclosed in “Monoclonal Antibodies: A manual of techniques”, H Zola (CRC Press, 1988) and in “Monoclonal Hybridoma Antibodies: Techniques and applications”, J G R Hurrell (CRC Press, 1982), both of which are incorporated herein by reference.

When potential binding molecules are selected from libraries, one or more selector peptides having defined motifs are usually employed. Amino acid residues that provide structure, decreasing flexibility in the peptide or charged, polar or hydrophobic side chains allowing interaction with the binding molecule may be used in the design of motifs for selector peptides. For example:
(i) Proline may stabilise a peptide structure as its side chain is bound both to the alpha carbon as well as the nitrogen;
(ii) Phenylalanine, tyrosine and tryptophan have aromatic side chains and are highly hydrophobic, whereas leucine and isoleucine have aliphatic side chains and are also hydrophobic;
(iii) Lysine, arginine and histidine have basic side chains and will be positively charged at neutral pH, whereas aspartate and glutamate have acidic side chains and will be negatively charged at neutral pH;
(iv) Asparagine and glutamine are neutral at neutral pH but contain a amide group which may participate in hydrogen bonds;
(v) Serine, threonine and tyrosine side chains contain hydroxyl groups, which may participate in hydrogen bonds.

Typically, selection of binding molecules may involve the use of array technologies and systems to analyse binding to spots corresponding to types of binding molecules.

The one or more protein-binding moieties may comprise a detectable moiety. The detectable moiety may be selected from the group consisting of a fluorescent moiety, a luminescent moiety, a chemiluminescent moiety, a radioactive moiety and an enzymatic moiety.

In a further embodiment of the methods of the invention, step (b) may be performed using an assay comprising a second binding agent capable of binding to the one or more proteins, the second binding agent also comprising a detectable moiety. Suitable second binding agents are described in detail above in relation to the first binding agents.

Thus, the proteins of interest in the sample to be tested may first be isolated and/or immobilised using the first binding agent, after which the presence and/or relative amount of said biomarkers may be determined using a second binding agent.

In one embodiment, the second binding agent is an antibody or antigen-binding fragment thereof; typically a recombinant antibody or fragment thereof. Conveniently, the antibody or fragment thereof is selected from the group consisting of: scFv; Fab; a binding domain of an immunoglobulin molecule. Suitable antibodies and fragments, and methods for making the same, are described in detail above.

Alternatively, the second binding agent may be an antibody-like binding agent, such as an affibody or aptamer.

Alternatively, where the detectable moiety on the protein in the sample to be tested comprises or consists of a member of a specific binding pair (e.g. biotin), the second binding agent may comprise or consist of the complimentary member of the specific binding pair (e.g. streptavidin).

Where a detection assay is used, it is preferred that the detectable moiety is selected from the group consisting of: a fluorescent moiety; a luminescent moiety; a chemiluminescent moiety; a radioactive moiety; an enzymatic moiety. Examples of suitable detectable moieties for use in the methods of the invention are described above.

Preferred assays for detecting serum or plasma proteins include enzyme linked immunosorbent assays (ELISA), radioimmunoassay (RIA), immunoradiometric assays (IRMA) and immunoenzymatic assays (IEMA), including sandwich assays using monoclonal and/or polyclonal antibodies. Exemplary sandwich assays are described by David et al in U.S. Pat. Nos. 4,376,110 and 4,486,530, hereby incorporated by reference. Antibody staining of cells on slides may be used in methods well known in cytology laboratory diagnostic tests, as well known to those skilled in the art.

Thus, in one embodiment the assay is an ELISA (Enzyme Linked Immunosorbent Assay) which typically involves the use of enzymes which give a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemiluminescent systems based on enzymes such as luciferase can also be used.

Conjugation with the vitamin biotin is frequently used since this can readily be detected by its reaction with enzyme-linked avidin or streptavidin to which it binds with great specificity and affinity.

In an alternative embodiment, the assay used for protein detection is conveniently a fluorometric assay. Thus, the detectable moiety of the second binding agent may be a fluorescent moiety, such as an Alexa fluorophore (for example Alexa-647).

Preferably, steps (b), (d), and/or (f) are performed using an array. The array may be a bead-based array or a surface-based array. The array may be selected from the group consisting of: macroarray; microarray; nanoarray.

In on embodiment, the method is for identifying agents capable of inducing a respiratory hypersensitivity response. Preferably, the hypersensitivity response is a humoral hypersensitivity response, for example, a type I hypersensitivity response. Preferably, the method is for identifying agents capable of inducing respiratory allergy.

In one embodiment, the population of dendritic cells or population of dendritic-like cells is a population of dendritic cells. Preferably, the dendritic cells are primary dendritic cells. Preferably, the dendritic cells are myeloid dendritic cells.

The population of dendritic cells or dendritic-like cells is preferably mammalian in origin. Preferably, the mammal is a rat, mouse, guinea pig, cat, dog, horse or a primate. Most preferably, the mammal is human.

In an embodiment the population of dendritic cells or population of dendritic-like cells is a population of dendritic-like cells, preferably myeloid dendritic-like cells.

In one embodiment, the dendritic-like cells express at least one of the markers selected from the group consisting of CD54, CD86, CD80, HLA-DR, CD14, CD34 and CD1a, for example, 2, 3, 4, 5, 6 or 7 of the markers. In a further embodiment, the dendritic-like cells express the markers CD54, CD86, CD80, HLA-DR, CD14, CD34 and CD1a.

In a further embodiment, the dendritic-like cells may be derived from myeloid dendritic cells. Preferably the dendritic-like cells are myeloid leukaemia-derived cells. Preferably, the myeloid leukaemia-derived cells are selected from the group consisting of KG-1, THP-1, U-937, HL-60, Monomac-6, AML-193, monocyte-derived dendritic cells (MoDC) and MUTZ-3. Most preferably, dendritic-like cells are MUTZ-3 cells. MUTZ-3 cells are human acute myelomonocytic leukemia cells that were available from 15 May 1995 under deposit number ACC 295 from Deutsche Sammlung für Mikroorganismen and Zellkulturen GmbH (DSMZ), Inhoffenstraβe 7B, Braunschweig, Germany (www.dsmz.de).

In one embodiment, the dendritic-like cells, after stimulation with cytokine, present antigens through CD1d, MHC class I and II and/or induce specific T-cell proliferation.

Hence, in one embodiment, the method is indicative of whether the test agent is or is not a respiratory sensitizing agent. In alternative or additional embodiment, the method is indicative of the respiratory sensitizing potency of the sample to be tested.

Thus, in one embodiment, the method is indicative of the sensitizer potency of the test agent (i.e., that the test agent is either, a non-sensitizer, a weak sensitizer, a moderate sensitizer, a strong sensitizer or an extreme sensitizer). The decision value and distance in PCA correlates with sensitizer potency.

Alternatively or additionally, test agent potency may be determined by, in step (e), providing:
(i) one or more extreme respiratory sensitizer positive control agent;
(ii) one or more strong respiratory sensitizer positive control agent;
(iii) one or more moderate respiratory sensitizer positive control agent; and/or
(iv) one or more weak respiratory sensitizer positive control agent,
   wherein the test agent is identified as an extreme respiratory sensitizer in the event that the presence and/or amount in the test sample of the one or more biomarker measured in step (b) corresponds to the presence and/or amount in the extreme positive control sample (where present) of the one or more biomarker measured in step (f); and/or is different from the presence and/or amount in the strong, moderate, weak and/or negative control sample (where present) of the one or more biomarkers measured in step (f),

wherein the test agent is identified as a strong respiratory sensitizer in the event that the presence and/or amount in the test sample of the one or more biomarker measured in step (b) corresponds to the presence and/or amount in the strong positive control sample (where present) of the one or more biomarker measured in step (f); and/or is different from the presence and/or amount in the extreme, moderate, weak and/or negative control sample (where present) of the one or more biomarkers measured in step (f),

wherein the test agent is identified as a moderate respiratory sensitizer in the event that the presence and/or amount in the test sample of the one or more biomarker measured in step (b) corresponds to the presence and/or amount in the moderate positive control sample (where present) of the one or more biomarker measured in step (f); and/or is different from the presence and/or amount in the extreme, strong, weak and/or negative control sample (where present) of the one or more biomarkers measured in step (f), and

wherein the test agent is identified as a weak respiratory sensitizer in the event that the presence and/or amount in the test sample of the one or more biomarker measured in step (b) corresponds to the presence and/or amount in the weak positive control sample (where present) of the one or more biomarker measured in step (f); and/or is different from the presence and/or amount in the extreme, strong, moderate and/or negative control sample (where present) of the one or more biomarkers measured in step (f).

Hence, step (e) may comprise or consist of providing the following categories of respiratory sensitizer positive control:
(a) extreme, strong, moderate and weak;
(b) strong, moderate and weak;
(c) extreme, moderate and weak;
(d) extreme, strong and moderate;
(e) extreme and strong;
(f) strong and moderate;
(g) moderate and weak;
(h) strong and weak;
(i) extreme and moderate;
(j) extreme and weak;
(k) extreme;
(l) strong;
(m) moderate;
(n) weak.

Negative and positive controls may be classified as respiratory non-sensitizers or respiratory sensitizers, respectively, based on clinical observations in humans.

Alternatively or additionally the method may comprise comparing the expression of the one or more biomaker measured in step (b) with one or more predetermined reference value representing the expression of the one or more biomarker measured in step (c) and/or step (e).

Generally, respiratory sensitizing agents are determined with an ROC AUC of at least 0.55, for example with an ROC AUC of at least, 0.60, 0.65, 0.70, 0.75, 0.80, 0.85, 0.90, 0.95, 0.96, 0.97, 0.98, 0.99 or with an ROC AUC of 1.00. Preferably, skin sensitizing agents are determined with an ROC AUC of at least 0.85, and most preferably with an ROC AUC of 1.

Typically, agents capable of inducing respiratory sensitization are identified using a support vector machine (SVM), such as those available from http://cran.r-project.org/web/packages/e1071/index.html (e.g. e1071 1.5-24). However, any other suitable means may also be used. SVMs may also be used to determine the ROC AUCs of biomarker signatures comprising or consisting of one or more Table 1 biomarkers as defined herein.

Support vector machines (SVMs) are a set of related supervised learning methods used for classification and regression. Given a set of training examples, each marked as belonging to one of two categories, an SVM training algorithm builds a model that predicts whether a new example falls into one category or the other. Intuitively, an SVM model is a representation of the examples as points in space, mapped so that the examples of the separate categories are divided by a clear gap that is as wide as possible. New examples are then mapped into that same space and predicted to belong to a category based on which side of the gap they fall on.

More formally, a support vector machine constructs a hyperplane or set of hyperplanes in a high or infinite dimensional space, which can be used for classification, regression or other tasks. Intuitively, a good separation is achieved by the hyperplane that has the largest distance to the nearest training datapoints of any class (so-called functional margin), since in general the larger the margin the lower the generalization error of the classifier. For more information on SVMs, see for example, Burges, 1998, Data Mining and Knowledge Discovery, 2:121-167.

In one embodiment of the invention, the SVM is ‘trained’ prior to performing the methods of the invention using biomarker profiles of known agents (namely, known sensitizing or non-sensitizing agents). By running such training samples, the SVM is able to learn what biomarker profiles are associated with agents capable of inducing sensitization. Once the training process is complete, the SVM is then able to predict whether or not the biomarker sample tested is from a sensitizing agent or a non-sensitizing agent.

Decision values for individual SVMs can be determined by the skilled person on a case-by-case basis. In one embodiment, the test agent is classified as a respiratory sensitizer if one or more test (or replicate thereof) have an SVM decision value of >0. In one embodiment, the test agent is classified as a non-respiratory sensitizer if one or more test (or replicate thereof) have an SVM decision value of 50.

In one embodiment, the method is for identifying agents that are respiratory sensitizers regardless of whether they are also skin sensitizers. In one embodiment, the method is for identifying agents that are respiratory sensitizers but are not skin sensitizers. In another embodiment, the method is for identifying agents that are respiratory sensitizers and skin sensitizers.

This allows test agents to be classified as sensitizing or non-sensitizing. Moreover, in one embodiment, by training the SVM with sensitizing agents of known potency (i.e. non-sensitizing, weak, moderate, strong or extreme sensitizing agents), the potency of test agents can also be identified comparatively.

However, this training procedure can be by-passed by pre-programming the SVM with the necessary training parameters. For example, agents capable of inducing sensitization can be identified according to the known SVM parameters using the SVM algorithm detailed in Table 5, based on the measurement of all the biomarkers listed in Table A and/or the expression data listed in Table B.

It will be appreciated by skilled persons that suitable SVM parameters can be determined for any combination of the biomarkers listed Table A by training an SVM machine with the appropriate selection of data (i.e. biomarker measurements from cells exposed to known sensitizing and/or non-sensitizing agents). Alternatively, the

Table A biomarkers may be used to identify agents capable of inducing respiratory sensitization according to any other suitable statistical method known in the art.

Alternatively, the Table A data and/or Table B data may be used to identify agents capable of inducing respiratory sensitization according to any other suitable statistical method known in the art (e.g., ANOVA, ANCOVA, MANOVA, MANCOVA, Multivariate regression analysis, Principal components analysis (PCA), Factor analysis, Canonical correlation analysis, Canonical correlation analysis, Redundancy analysis Correspondence analysis (CA; reciprocal averaging), Multidimensional scaling, Discriminant analysis, Linear discriminant analysis (LDA), Clustering systems, Recursive partitioning and Artificial neural networks).

Preferably, the method of the invention has an accuracy of at least 65%, for example 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% accuracy.

Preferably, the method of the invention has a sensitivity of at least 65%, for example 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sensitivity.

Preferably, the method of the invention has a specificity of at least 65%, for example 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% specificity.

By “accuracy” we mean the proportion of correct outcomes of a method, by “sensitivity” we mean the proportion of all positive chemicals that are correctly classified as positives, and by “specificity” we mean the proportion of all negative chemicals that are correctly classified as negatives.

In one embodiment, the method of the first aspect of the invention comprises concurrently or consecutively performing a method for identifying agents capable of inducing sensitization of mammalian skin described in PCT publication number WO 2012/056236 which is incorporated herein by reference (in particular, the aspects and embodiments of the invention, as well as the claims). Preferably the method for identifying agents capable of inducing sensitization of mammalian skin is performed concurrently with the method of the first aspect of the present invention (i.e., determining whether a test compound is a skin and/or respiratory sensitizer by measuring relevant marker expression in the same cell sample(s) exposed to the test agent).

A second aspect of the invention provides an array for use in the method of the first aspect of the invention (or any embodiment or combination of embodiments thereof), the array comprising or consisting of one or more binding moieties as defined above. In one embodiment, the binding moieties are (collectively) capable of binding to all of the biomarkers defined in Table A(i). In a further embodiment, the binding moieties are (collectively) capable of binding to all of the biomarkers defined in Table A(ii). In a still further embodiment, the binding moieties are (collectively) capable of binding to all of the biomarkers defined in Table A(iii). Preferably, the binding moieties are (collectively) capable of binding to all of the biomarkers defined in Table A.

The binding moieties may be immobilised.

Arrays per se are well known in the art. Typically they are formed of a linear or two-dimensional structure having spaced apart (i.e. discrete) regions (“spots”), each having a finite area, formed on the surface of a solid support. An array can also be a bead structure where each bead can be identified by a molecular code or colour code or identified in a continuous flow. Analysis can also be performed sequentially where the sample is passed over a series of spots each adsorbing the class of molecules from the solution. The solid support is typically glass or a polymer, the most commonly used polymers being cellulose, polyacrylamide, nylon, polystyrene, polyvinyl chloride or polypropylene. The solid supports may be in the form of tubes, beads, discs, silicon chips, microplates, polyvinylidene difluoride (PVDF) membrane, nitrocellulose membrane, nylon membrane, other porous membrane, non-porous membrane (e.g. plastic, polymer, perspex, silicon, amongst others), a plurality of polymeric pins, or a plurality of microtitre wells, or any other surface suitable for immobilising proteins, polynucleotides and other suitable molecules and/or conducting an immunoassay. The binding processes are well known in the art and generally consist of cross-linking covalently binding or physically adsorbing a protein molecule, polynucleotide or the like to the solid support. Alternatively, affinity coupling of the probes via affinity-tags or similar constructs may be employed. By using well-known techniques, such as contact or non-contact printing, masking or photolithography, the location of each spot can be defined. For reviews see Jenkins, R. E., Pennington, S. R. (2001, Proteomics, 2,13-29) and Lal et al (2002, Drug Discov Today 15;7(18 Suppl):S143-9).

Typically the array is a microarray. By “microarray” we include the meaning of an array of regions having a density of discrete regions of at least about 100/cm², and preferably at least about 1000/cm². The regions in a microarray have typical dimensions, e.g. diameter, in the range of between about 10-250 μm, and are separated from other regions in the array by about the same distance. The array may alternatively be a macroarray or a nanoarray.

Once suitable binding molecules (discussed above) have been identified and isolated, the skilled person can manufacture an array using methods well known in the art of molecular biology.

Preferably, the arrays is consistent with the method described in the first aspect of the invention, and one or more of the embodiments described therein.

A third aspect of the present invention provides the use of one or more (preferably two or more) biomarkers selected from the group defined in Table A(i) Table A(ii) and/or Table A(iii) in combination for identifying hypersensitivity response sensitising agents. Preferably, all of the biomarkers defined in Table A(i) and Table A(ii) are used collectively for identifying hypersensitivity response sensitising agents. Preferably, the use is consistent with the method described in the first aspect of the invention, and one or more of the embodiments described therein.

A fourth aspect of the present invention provides the use of one or more (preferably two or more) binding moieties as defined in the first aspect of the invention. Preferably, binding moieties for all of the biomarkers defined in Table A(i) and Table A(ii) are used collectively for identifying hypersensitivity response sensitising agents. Preferably, the use is consistent with the method described in the first aspect of the invention, and one or more of the embodiments described therein.

A fifth aspect of the invention provides an analytical kit for use in a method according the first aspect of the invention, comprising or consisting of:
A) an array according to the second aspect of the invention and/or one or more binding moiety as defined in the first aspect of the invention; and
B) instructions for performing the method according to the first aspect of the invention (optional).

The analytical kit may comprise one or more control agents. Preferably, the analytical kit comprises or consists of the above features, together with one or more negative control agents and/or one or more positive control agents as defined in the first aspect of the invention. Preferably, the analytical kit is consistent with the method described in the first aspect of the invention, and one or more of the embodiments described therein.

A sixth aspect of the invention provides a method of treating or preventing a respiratory type I hypersensitivity reaction (such as respiratory asthma) in a patient comprising the steps of:
(a) providing one or more test agent that the patient is or has been exposed to;
(b) determining whether the one or more test agent provided in step (a) is a respiratory sensitizer using a method provided in the first aspect of the present invention; and
(c) where one or more test agent is identified as a respiratory sensitizer, reducing or preventing exposure of the patient to the one or more test agent identified as a respiratory sensitizer and/or providing appropriate treatment for the symptoms of sensitization.

Preferably, the one or more test agent that the patient is or has been exposed to is an agent that the patient is presently exposed to at least once a month, for example, at least once every two weeks, at least once every week, or at least once every day.

Treatments of the symptoms of sensitization may include short-acting beta2-adrenoceptor agonists (SABA), such as salbutamol; anticholinergic medications, such as ipratropium bromide; other adrenergic agonists, such as inhaled epinephrine; Corticosteroids such as beclomethasone; long-acting beta-adrenoceptor agonists (LABA) such as salmeterol and formoterol; leukotriene antagonists such as montelukast and zafirlukast; and/or mast cell stabilizers (such as cromolyn sodium) are another non-preferred alternative to corticosteroids.

Preferably, the method of treatment is consistent with the method described in the first aspect of the invention, and one or more of the embodiments described therein.

A seventh aspect of the invention provides a computer program for operating the method of the first aspect of the invention, for example, for interpreting the expression data of step (b), (d) and/or (f) and thereby determining whether one or more test agent is a respiratory sensitizer. The computer program may be a programmed SVM. The computer program may be recorded on a suitable computer-readable carrier known to persons skilled in the art. Suitable computer-readable-carriers may include compact discs (including CD-ROMs, DVDs, Blue Rays and the like), floppy discs, flash memory drives, ROM or hard disc drives. The computer program may be installed on a computer suitable for executing the computer program.

Preferred, non-limiting examples which embody certain aspects of the invention will now be described, with reference to the following figures:

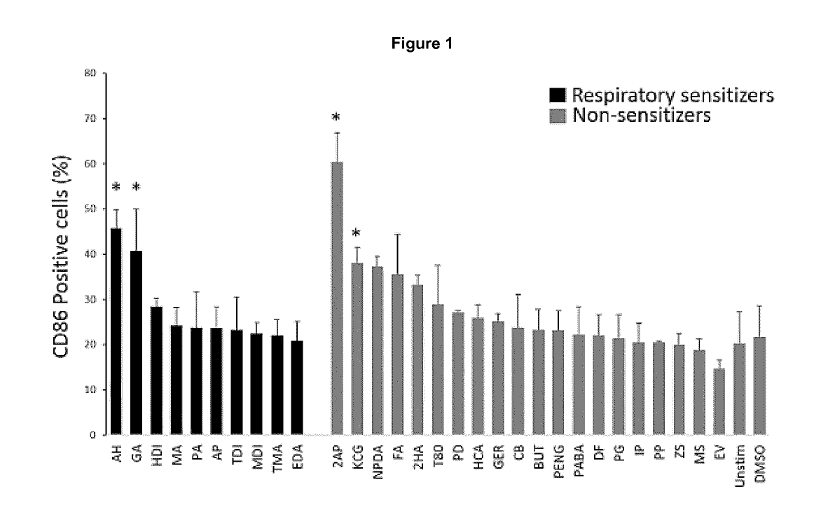

FIG. 1. CD86 expression of MUTZ-3 cells following chemical stimulations. Data shown is an average of chemical stimulations, (n=3), 4-Aminobenzoic acid, DMSO and unstimulated cells (n=6) and potassium permanganate, 2-aminophenol, Hexylcinnamic aldehyde and 2-Hydroxyethyl acrylate (n=2), with error bars showing standard deviation. Statistical significance was determined by student's t-test, comparing each stimulation with its corresponding vehicle, with p<0.05 indicated by *.

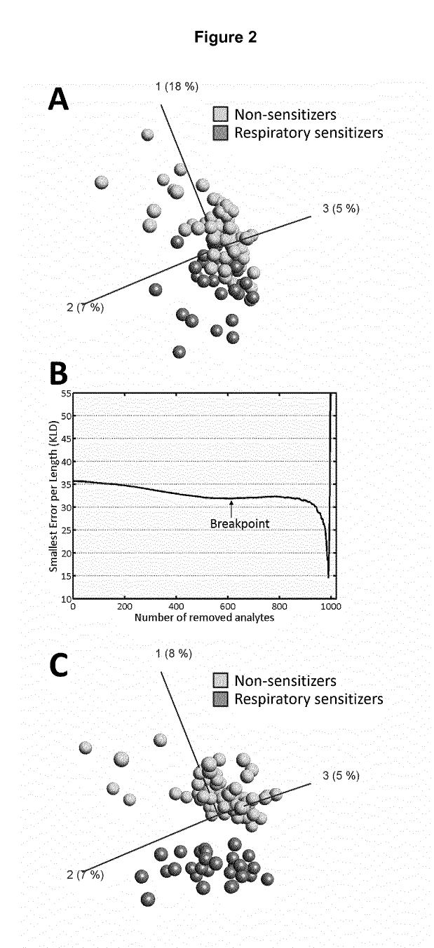

FIG. 2. Establishment of a predictive biomarker signature for prediction of respiratory sensitization. (A) Unsupervised learning was used to construct the representation of the dataset. The method was visualized using PCA based on 999 transcripts identified by one-way ANOVA p-value filtering between respiratory sensitizers (blue, n=29) and non-respiratory sensitizers (green, n=74). (B) The 999 transcripts identified by p-value filtering were used as input into an algorithm for backward elimination. A breakpoint in Kullback-Leibler divergence was observed after removal of 610 transcripts. (C) The remaining 389 transcripts were used as input variables into a PCA. As illustrated in the figure, a complete seperation between respiratory sensitizers and non-respiratory sensitizers was achieved in the training data.

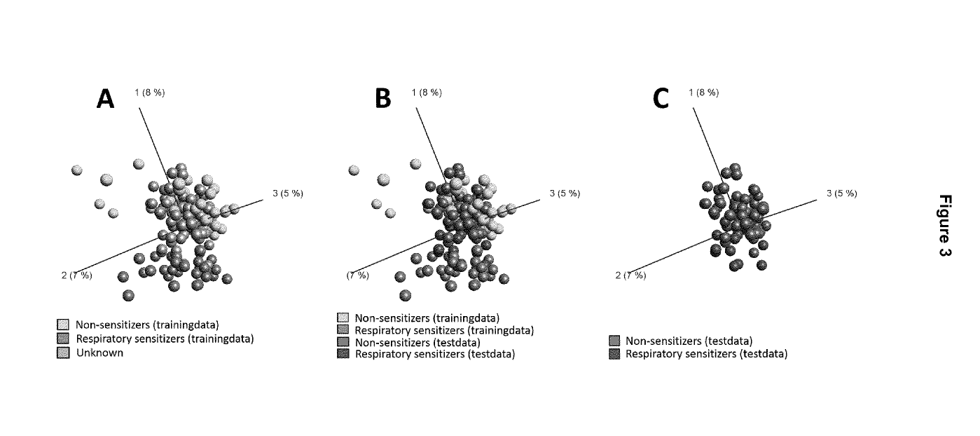

FIG. 3. Visual classification of independent test compounds using GARD

Respiratory Prediction Signature, GRPS. (A) The PCA space was constructed from the three first PCA components from the panel of reference chemicals (n=103) used for biomarker signature identification, using the 389 genes of GRPS as input into the unsupervised representation. Each of the chemicals in the test dataset (n=92) were plotted into the PCA space without allowing the compounds to influence PCA components. (B) Samples in the test dataset was colored according to sensitizing properties as either respiratory sensitizers (dark blue) or non-respiratory sensitizers (dark green). A separation between respiratory sensitizers and non-respiratory sensitizers can be seen along the first PCA component for both the training data and the test data. (C) The training dataset has been removed in order to obtain a clear view of the training dataset.

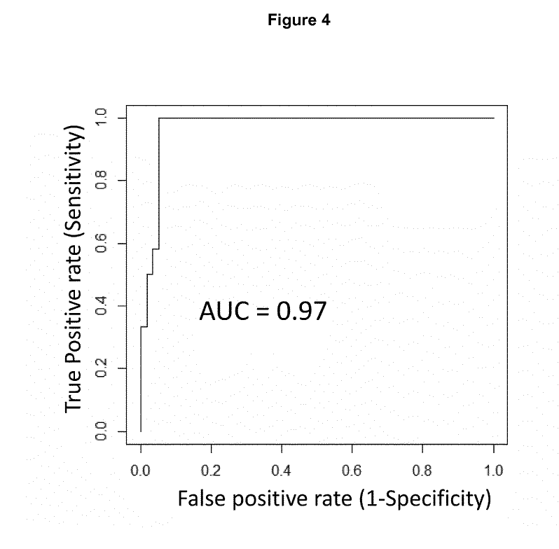

FIG. 4. Support Vector Machine (SVM) classifications of the test dataset. The predictor performance of GRPS was validated using SVM for supervised machine learning. The SVM algorithm was inductively learned by experience to the compounds in the training dataset (n=103) and subsequently applied to predict each individual sample in the test dataset (n=70, vehicle controls excluded). The predictive performance was evaluated by ROC curve analysis and estimated to an Area Under the Curve (AUC) of 0.97.

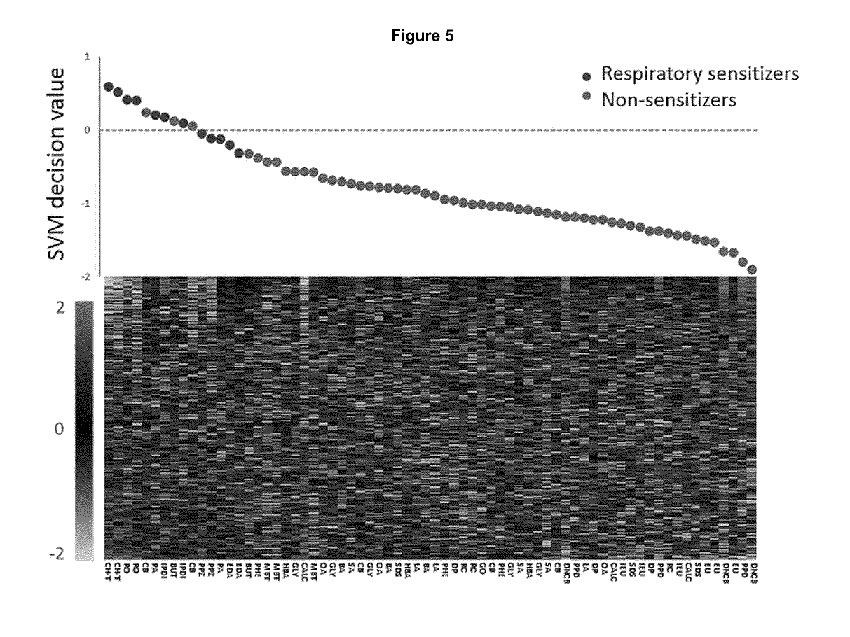

FIG. 5. Classification and gene expression of respiratory sensitizers in the independent test dataset. The SVM algorithm was once again trained on the samples in the training dataset (n=103) and subsequently applied in order to classify samples in the independent test dataset (n=70, vehicle controls were excluded). SVM decision values for each individual sample in the independent test dataset were plotted in decreasing order and coloured according to sensitizing capacity (Respiratory sensitizers=purple, non-respiratory sensitizers=dark green). The dotted line in the scatterplot represents the threshold level for classifications as respiratory sensitizers (SVM decision value>0) or non-respiratory sensitizers (SVM decision value<0). Relative expression of transcripts within GRPS is shown in the heat map.

### EXAMPLES

Introduction

Background: Repeated exposure to certain low molecular weight (LMW) chemical compounds may result in development of allergic reactions in the skin or in the respiratory tract. In most cases, a certain LMW compound selectively sensitize the skin, giving rise to allergic contact dermatitis (ACD), or the respiratory tract, giving rise to occupational asthma (OA). To limit occurrence of allergic diseases, efforts are currently being made to develop predictive assays that accurately identify chemicals capable of inducing such reactions. However, while a few promising methods for prediction of skin sensitization have been described, to date no validated method, in vitro or in vivo, exists that is able to accurately classify chemicals as respiratory sensitizers.

Results: Recently, we presented the in vitro based Genomic Allergen Rapid Detection (GARD) assay as a novel testing strategy for classification of skin sensitizing chemicals based on measurement of a genomic biomarker signature. We have expanded the applicability domain of the GARD assay to classify also respiratory sensitizers by identifying a separate biomarker signature containing 389 differentially regulated genes for respiratory sensitizers in comparison to non-respiratory sensitizers. By using an independent data set in combination with supervised machine learning, we validated the assay, showing that the identified genomic biomarker is able to accurately classify respiratory sensitizers.

Conclusions: We have identified a genomic biomarker signature for classification of respiratory sensitizers. Combining this newly identified biomarker signature with our previously identified biomarker signature for classification of skin sensitizers, we have developed a novel in vitro testing strategy with a potent ability to predict both skin and respiratory sensitization in the same sample.

Materials and Methods

Chemicals

A panel of 32 reference chemicals comprising a selection of 10 well characterized respiratory sensitizers and 22 non-respiratory sensitizers, collectively termed the training dataset, were used for cell stimulations in order to establish the predictive genomic biomarker signature. The respiratory sensitizers were ammonium hexachloroplatinate, ammonium persulfate, ethylenediamine, glutaraldehyde, hexamethylene diisocyanate, maleic anhydride, methylene diphenyl diisocyanate, phtalic anhydride, toluene diisocyanate and trimellitic anhydride. The non-respiratory sensitizers were 1-butanol, 2-aminophenol, 2-hydroxyethyl acrylate, 2-nitro-1,4-phenylenediamine, 4-aminobenzoic acid, chlorobenzene, dimethyl formamide, ethyl vanillin, formaldehyde, geraniol, hexylcinnamic aldehyde, isopropanol, Kathon CG, methyl salicylate, penicillin G, potassium dichromate, potassium permanganate, propylene glycol, Tween 80, zinc sulfate and the vehicle controls dimethyl sulfoxide and water. Additionally, a panel of 25 chemicals, including 6 respiratory sensitizers and 19 non-respiratory sensitizers, collectively termed the independent test dataset, were used for cell stimulations in order to form an independent testset for validation of the identified predictive genomic biomarker signature. The independent training dataset comprised both control chemicals, included during the training of the model, as well as chemicals previously unseen during training of the model. The respiratory sensitizers were chloramine T, ethylenediamine, Isophorone diisocyanate, phtalic anhydride, piperazine and reactive orange. The non-respiratory sensitizers were 1-butanol, 2,4-dinitrochlorobenzene, 2-mercaptobenzothiazole, benzaldehyde, chlorobenzene, cinnamyl alcohol, diethyl phthalate, eugenol, glycerol, glyoxal, isoeugenol, lactic acid, octanoic acid, phenol, p-hydroxybenzoic acid, p-phenylendiamine, resorcinol, salicylic acid and sodium dodecyl sulfate. All chemicals were purchased from Sigma-Aldrich (St. Louis, Mo., USA). Chemicals were dissolved and diluted into GARD input concentration in either water or DMSO prior to stimulation of cells. For chemicals dissolved in DMSO, the in-well concentration of DMSO was 0.1%. Monitoring of chemical cytotoxicity and establishment of GARD input concentration for each chemical compound was performed as previously described [41,42]. In short, GARD input concentration was determined according to the following decision schedule: Non-toxic and freely soluble compounds were used at a concentration corresponding to 500 uM. Non-toxic and poorly soluble compounds, insoluble at 500 uM, were used at highest soluble concentration. Toxic compounds were used at a concentration yielding 90% relative viability (Rv90). The criterion that was first met determined the GARD input concentration for each compound. The GARD input concentration, sensitizing potency and solvent are presented in Table 1 for compounds used to establish the predictive genomic biomarker signature, and in Table 2 for compounds used to validate the predictive genomic biomarker signature.

Cell Cultures, Phenotypic Analysis, Chemical Exposure, Cell Harvest and Mrna Isolation

The human acute myelomonocytic leukemia cell line MUTZ-3[68,69] was obtained from Leibniz-lnstitut DSMZ-Deutsche Sammlung von Mikroorganismen and Zellkulturen (DSMZ, Braunschweig, Germany). Maintenance of cells, chemical stimulation of MUTZ-3 and all subsequent isolation of mRNA and preparation of cDNA were performed as previously described [41,42]. In short, a phenotypic control of MUTZ-3 was performed using flow cytometry prior to chemical stimulation to ensure cells were in an immature state. The following FITC-conjugated mouse monoclonal antibodies (mAbs) were used: CD1a (DakoCytomation, Glostrup, Denmark), CD34, CD86, HLA-DR, IgG1 (BD Biosciences, Franklin Lakes, N.J.). The following PE-conjugated mouse monoclonal antibodies were used: CD14 (DakoCytomation), CD54, CD80, IgG1 (BD Biosciences). Cell viability was determined using Propidium Iodide (BD Biosciences) staining. Samples were run on FACSCanto II instrument. Data was acquired using FACS Diva software (BD Biosciences) and analyzed using FCS Express V4 (De Novo Software, Los Angeles, Calif.). Gating was performed to exclude cell debris and non-viable cells based on forward- and side-scattering properties and quadrants established using isotype-controls. During chemical exposure, cells were seeded at 200.000 cells/ml in 24-well plates and exposed to chemical compound at the GARD input concentration. Stimulated cells were harvested after 24 h incubation at 37° C., 5% CO₂ and RNA was isolated with TRIzol® reagent (Life Technologies, Carlsbad, Calif.) using standardized protocols provided by the manufacturer. In parallel, a control of the maturity state of the cells was performed by flow cytometric analysis of CD86. Preparation of cDNA and hybridization, washing and scanning of the Human Gene 1.0 ST Arrays (Affymetrix, Santa Clara, Calif., USA) was performed, according to standardized protocols provided by the manufacturer (Affymetrix).

Microarray Data Analysis and Statistical Methods

Gene expression data obtained from the Human Gene 1.0 ST Arrays were normalized using the Single-Channel array normalization (SCAN) algorithm [70], and potential batch effects between different set of experiments were adjusted using the ComBat [71] empirical bayes method. Normalizations and batch adjustments were performed in R statistical software [72] using the open software Bioconductor v2.14 [73] with the additional software packages SCAN.UPC [70] and sva [74] . Normalized data was imported into Qlucore Omics Explorer 3.0 (Qlucore AB, Lund, Sweden) and visualized using Principal Component Analysis (PCA) [75]. Predictors were selected from a one-way ANOVA p-value filtration, using false discovery rate (FDR) [76] to adjust for multiple hypothesis testing, comparing respiratory sensitizers and non-respiratory sensitizers. A wrapper algorithm for Backward Elimination [41,52] was applied on the top 999 predictors, to further reduce and refine the biomarker signature size. The Backward Elimination algorithm was modified to minimize the Kullback-Leibler error [53] rather than maximizing the Area Under the Receiver Operating Characteristic (AUC ROC) [77], in order to enable signature optimization in cases where the AUC ROC reaches 1.0. The selected top 389 predictors after backward elimination were collectively designated “GARD Respiratory Prediction Signature”. The script for Backwards Elimination was programmed in R, with the additional package e1071 [78]. The method by which the predictive genomic biomarker signature was established was validated using cross-validation based on Support Vector Machines (SVM) [79], based on a linear kernel, as described previously [41]. In short, the training dataset was randomly divided into a new cross-validation training dataset comprising 70% of the stimulations, and a cross-validation test dataset comprising 30% of the stimulations. In addition, care was taken to maintain the same proportion between respiratory sensitizers and non-respiratory sensitizers as in the complete training dataset. A new predictive genomic biomarker signature was identified from the cross-validation training dataset using one-way ANOVA p-value filtration as described above. The identified predictive biomarker signature was used to train a SVM based on the information in the cross-validation training dataset. SVMs were compiled in R statistical software with the additional package e1071. The SVM model was subsequently used to predict the samples of the cross-validation test dataset. The process of biomarker identification was repeated 20 times and the robustness of the feature selection process was evaluated by calculating the frequency (referred to as the Validation call frequency, of VCF) by which each individual transcript was included in the 20 training datasets. The predictive performance of the GRPS in terms of prediction of unknown samples was estimated using the independent test dataset as described in [46]. In short, a SVM was trained on the training dataset, using the GRPS as variable input. Subsequently, the SVM was then used to predict the samples in the training dataset in the same way as described for the cross-validation above, and the predictive performance of the model was evaluated using AUC ROC, determined in R statistical environment using the additional package ROCR [80]. Classification of samples as respiratory sensitizers or non-respiratory sensitizers were based on SVM decision values on replicate level. Hence, a chemical was classified as a respiratory sensitizer if any of the replicate stimulations from a certain chemical stimulation had an SVM decision value >0. The accuracy, sensitivity and selectivity of the assay was determined using cooper statistics [81]. The biological relevance of the GRPS was explored using MetaCore™ (Thomson Reuters, New York, N.Y.) by performing a functional enrichment. The top 999 predictors from a p-value filtering were used as input into the MetaCore™ algorithm and biological relevance was established by exploring the Canonical Pathways associated with input molecules. The array data has been uploaded to ArrayExpress (http://www.ebi.ac.uk/arrayexpress/) with accession number E-MEXP-3773.

Results

Phenotypic Analysis of Unstimulated and Chemically Stimulated MUTZ-3 Cells

Prior to chemical challenge, cells were quality controlled by measuring the cellular expression of common myeloid and dendritic cell markers using flow cytometry. These markers included CD1a, CD14, CD34, CD54, CD80, CD86 and HLA-DR. Results correlated with previously published phenotypic profiles [41], ensuring that cells were successfully maintained in an immature state. Following chemical stimulation, using a panel of reference chemicals comprising 10 respiratory sensitizers and 20 non-respiratory sensitizers, as well as vehicle controls (Table 1), the general maturity state of the cells was again verified by measuring levels of cell surface expression of the co-stimulatory marker CD86, with results presented in FIG. 1. Chemically induced up-regulation of CD86 for each stimulation in comparison to unstimulated cells was evident in cells after a number of stimulations. However, due to large standard deviation between replicate stimulations, only regulation induced by the respiratory sensitizers ammonium hexachloroplatinate and glutaraldehyde could be confirmed as statistically significant (students t-test, p<0.05). Consequently, an assay using CD86 as a single biomarker for classification of respiratory sensitization would result in a sensitivity of only 20%. Additionally, up-regulation of CD86 was also observed in the non-respiratory sensitizing stimulations 2-aminophenol and kathon CG. Thus, we concluded that CD86 could not be used as a single biomarker in the MUTZ-3 cell line to classify respiratory chemical sensitizers. However, as many of the respiratory sensitizers have a poor solubility in cell media, and can thus not be used in sufficient concentration to induce cytotoxicity, the expression of CD86 may act as a complementary quality control to ensure bioavailability of chemicals stimulations.

Identification of a Predictive Genomic Biomarker Signature by Transcriptional Profiling of Chemically Stimulated MUTZ-3

Chemically induced changes in the MUTZ-3 cells were investigated on transcriptional-wide basis in order to identify the most discriminatory transcripts between respiratory sensitizers and non-respiratory sensitizers. Following 24 h of cellular stimulation with a panel of reference chemicals, mRNA was collected for transcriptional profiling. The stimulations included 10 different respiratory sensitizers, 20 non-respiratory sensitizers (negative controls) and vehicle controls (DMSO, distilled water). All stimulations were performed in biological triplicates except for 4-aminobenzoic acid, which was analyzed in 6 replicates due to internal controls, and potassium permanganate, which was analyzed in only 2 replicates due to a faulty array. In addition, vehicle controls (DMSO and distilled water) were analyzed in 6 replicates each. Quality control of samples revealed that one of the replicate stimulations of ammonium persulfate was a significant outlier and had to be removed in order not to interfere with biomarker discovery. Summarized, the data set ready for analysis consisted of 103 arrays, each with measurements of 29,141 transcripts.

Gene expression data was imported into Qlucore Omics Explorer and visualized using principal component analysis (PCA). We applied a tiered approach for feature selection, combining a filtering method in order to reduce the noise in the dataset and select predictors based on intrinsic properties, and a wrapper method based on Backward Elimination in order to reduce the number of genes in the signature by taking into account how each individual predictor performs collectively with the entire signature. The filtering method was based entirely on p-values, as determined by one-way ANOVA analysis, comparing respiratory sensitizers and non-respiratory sensitizers. The wrapper method was based on repeated supervised learning. The algorithm for Backward Elimination was developed in house [52] and iteratively extracted a subset of transcripts which were subsequently evaluated by training and testing of a Support Vector machine (SVM), using a leave-one-out cross validation procedure. The least informative variables were removed, and the process was repeated until the highest performance of classification model was achieved. Due to computational limits, approximately 1000 genes is an appropriate amount of potential predictors to use as an input in the algorithm for Backward Elimination. In the present data set, this pre-selection of predictor candidates resulted in 999 genes, with a p-value of 0.024 or lower. As illustrated in FIG. 2A, these genes were collectively able to achieve a partition between respiratory sensitizers and non-respiratory sensitizers, although some overlap occurred between the two groups. Reducing the number of predictors further, by the ranking given by their p-value, did not achieve a clear separation, even though the data contained predictor candidates with p-values down to 10⁻⁶. The in house developed algorithm for Backward Elimination was then applied, removing the predictors (genes) that contribute the least information. A local minimum in Kullbach-Liebler Divergence (KLD) [53] was observed when 610 predictors were eliminated (FIG. 2B). The remaining 389 genes are collectively termed the “GARD Respiratory Prediction Signature” (GRPS), and their ability to differentiate between respiratory chemical sensitizers and non-respiratory sensitizers in the training dataset are illustrated in FIG. 2C. The identities of the genes are listed in Table A. In order to validate the method by which the biomarker signature was established, we performed a cross validation procedure where we randomly divided the samples used during biomarker discovery into a training set and a test set as described in methods. The process was iterated 20 times and the frequency of each predictor transcript among the 20 new biomarker signatures was used as a measurement of robustness. Results from this exercise were tabulated as Validation Call Frequencies (VCF) and are summarized in table A.

Visual Classification of Independent Test Compounds Using GARD Respiratory Prediction Signature as an Estimate of Predictive Performance

The predictive performance of GRPS was validated using an independent test dataset comprising both respiratory sensitizers as well as non-respiratory sensitizers in order to illustrate the relevance of the genomic biomarker signature as a predictive assay for respiratory sensitizers. The chemical compounds included in the independent test dataset are described in Table 2. Some of the compounds used during the training of the model, including 1-butanol, chlorobenzene, ethylenediamine, phtalic anhydride, and the vehicle controls (DMSO, distilled water) were included also in the independent test dataset to be used as controls. The remaining chemicals were unseen by the model prior to classification of the samples. All chemicals in the independent test dataset were based on additional stimulations, separated in time from the stimulations comprising the training dataset. Therefore, both the unseen compounds, as well as the compounds seen by the model during the identification of the GRPS could be classified without the risk of over fitting the model. Following 24 h of cellular stimulation, mRNA was collected, converted to cDNA and hybridized to the microarrays. The stimulations included 6 respiratory sensitizers, 19 non-respiratory sensitizers and vehicle controls (DMSO, distilled water). The non-respiratory sensitizing stimulations were reused from a previous set of experiments [41] and performed in biological triplicates. Stimulations performed with the respiratory sensitizers, together with the non-respiratory sensitizer 1-butanol, comprised a novel round of stimulations, and were performed in biological duplicates. The chemical chlorobenzene was included in both sets due to internal controls, hence comprised a total of 5 stimulations. In addition, vehicle controls DMSO and distilled water were analyzed in 13 and 9 replicates, respectively. In summary, the independent test dataset comprised 92 arrays. The process of performing visualized classifications of unknown samples is sequentially illustrated in FIG. 3, using the test dataset to highlight the methodology. In an initial step, the training data set, i.e. the panel of reference chemicals used to identify the predictive GRPS genomic biomarker signature, was first used to generate the PCA space, using the 389 genes in the GRPS as variable input. The PCA was then frozen in space, and each of the compounds in the test dataset was plotted into this space without allowing them to influence the PCA components (Fig.3A). As demonstrated in FIG. 3B, upon identification of true identities of the samples in the test dataset, a clear separation between respiratory sensitizers and non-respiratory sensitizers can be seen along the first PCA component for both the training data and the test data, indicating that similarities and differences in structure of gene expression in the GPRS between respiratory sensitizers and non-respiratory sensitizers was present also in the test dataset. FIG. 3C illustrates the test dataset plotted into the frozen PCA space generated by the training dataset, but where the training dataset have been removed in order to facilitate interpretation. As seen in the figure, respiratory sensitizers and non-respiratory sensitizers appears to be separated by a hyper plane generated by the 2^{nd} and 3^{rd} principal components, indicating that the GRPS clearly contain information relevant for achieving discrimination between respiratory sensitizers and non-respiratory sensitizers in the previously unseen samples in the independent test dataset.

SVM Classifications and Predictive Performance of GRPS

In a consecutive step to classify the samples in the independent test dataset, the visual classifications were challenged with a binary classification model, using an SVM for supervised machine learning. The SVM was trained on the training data set to recognize differences in gene expression structure between respiratory sensitizers and non-respiratory sensitizers within the GRPS. The trained SVM model was applied to classify each sample in the independent test data set, on the level of each individual replicate, as either a respiratory sensitizer or a non-respiratory sensitizer. The output from the SVM, the SVM decision values, were compared to true identities of samples in the test dataset, and the performance of the predictor was evaluated using ROC AUC analysis with results illustrated in FIG. 4. SVM classifications were based on linear kernels with an unbiased maximum-margin hyperplane separating the two groups, hence threshold for classifications as respiratory sensitizers corresponded to a SVM decision value >0. As shown in the figure, predictor performance of the GRPS on the level of individual stimulations was estimated to an area under the ROC curve of 0.97. Decision values obtained from the SVM classifications for each chemical compound in the test dataset are presented in Table 3 (n=70, vehicle controls excluded). Data presented in this table is further summarized in FIG. 5. In the figure, samples are sorted in a descending scale from highest to lowest SVM value, and the SVM decision value for each compound in the test dataset are correlated to the individual expression profile of the 389 transcripts in the GRPS. In order to facilitate interpretation, samples in the figure were colored according to sensitizing capacity (Respiratory sensitizers=purple, non-respiratory sensitizers=dark green) and the threshold value for classification is illustrated by the dotted line. As illustrated in the figure, although some overlap was observed, the majority of the respiratory sensitizers were assigned SVM decision values that were higher than corresponding values assigned to non-respiratory sensitizers, which also correlated with differences in the expression profiles between the majorities of compounds within each group. For decision making and classification on sensitizing capacity for each chemical, we chose to use the cut-off stating that any given sample in the test dataset should be classified as a respiratory sensitizer if any of the replicate stimulations has an SVM decision value >0, as determined in previously published protocols [42]. Based on this criterion, the accuracy, sensitivity and specificity of the GRPS was estimated using cooper statistics to 84%, 67%, and 89%, respectively.

Canonical Pathways Associated with Respiratory Sensitizers and GARD Prediction Signature

Aiming to investigate the biologic response initiated by respiratory chemical sensitizers in MUTZ-3 cells, the data was analyzed using functional enrichment analysis in Metacore™. The top 999 genes, selected with p-value filtering, were used as input into Metacore™. Of the 999 genes, Metacore^{TM} was able to map 948 to unique IDs. Significantly regulated pathways (p<0.01) are listed in Table 4, ranked by −log (p-Value) and sorted in order of statistical significance. Genes present in GRPS are indicated in bold. A clear majority of these identified and significantly regulated pathways are mainly driven by a limited set of molecules. The most highly populated pathways included oxidative phosphorylation (26 molecules) and Ubiquinone metabolism (19 molecules), showing that cellular events such as oxidation-reduction processes and the respiratory electron transport chain function is highly affected by the studied chemicals. In addition, several of the less significantly regulated pathways, including Inhibitory PD-1 signaling in T cells, Antigen presentation by MHC class I and MHC class II, Generation of memory CD4+ T cells, IL-33 signaling pathway are relevant from an immunological point of view. Of note, central for many of these pathways is the bridge between innate and adaptive immunity, and the engagement of innate immune responses initiated by recognition of foreign substances, leading to dendritic cell maturation and activation of specific T-cell responses. Key aspects of this process is well monitored and significantly regulated in the MUTZ-3 cell line, including upregulation of antigen presentation-associated molecules, such as MHC class I and MHC class II complex, upregulation of co-stimulatory molecules, such as CD80 and CD86, and cross-talk with key players such as T-cells through initiation and coordination of pathways responsible for driving the immune response. Of note, activated pathways are only to a very limiting extent overlapping with pathways activated by skin sensitizers in MUTZ-3 [54] (Granzyme B and Granzyme A signaling), indicating that respiratory sensitizers and skin sensitizers are involved in engagement of different signaling pathways.

Discussion

A variety of chemicals are able of inducing allergic hypersensitivity reactions in both skin and respiratory tract, eventually giving rise to clinical symptoms of Allergic Contact Dermatitis (ACD) or Occupation Asthma (OA). Although the numbers of chemicals able of inducing respiratory sensitization are far fewer in comparison to those causing skin sensitization, identification and hazard classification of respiratory chemical sensitizers remains an area of great importance due to the severe impact on health and quality of life associated with acquired OA. Development of reliable assays that accurately identifies respiratory sensitizers as well as distinguishing those from skin sensitizers have proven challenging. In previous studies, we described the development and application of the Genomic Allergen Rapid Detection (GARD) assay as an in vitro alternative to animal testing for identification and risk assessment of skin sensitizing chemicals. In the GARD assay, unknown test chemicals are classified based on readout from a pre-determined genomic biomarker signature, measured by genome-wide transcriptional profiling. Utilizing the great versatility that comes with analyzing the complete transcriptome, we hypothesized that the applicability domain of GARD could be broadened to also cover hazard classification of respiratory sensitizers through the identification of an alternative genomic biomarker signature.

In the current study, we present a further development of GARD, allowing for classification of respiratory sensitizing chemicals, using a different biomarker signature termed the GARD Respiratory Prediction Signature (GRPS). The intended use of the defined GRPS will thus be in a novel combined in vitro assay, in which MUTZ-3 cells are stimulated with the unknown compounds to be classified. Of note, using the two distinct biomarker signatures, the compound can be classified as either a skin sensitizer, a respiratory sensitizer or a non-sensitizer. Chemicals that are able to induce both respiratory and skin sensitization will also be specifically classified as such.

The GRPS was identified, using a set of reference chemicals known to be either respiratory sensitizers or non-respiratory sensitizers. Differentially regulated genes in these two groups were then identified by an ANOVA p-value filtering and further optimized, using an in house developed wrapper algorithm for backward elimination. We suggest that the 389 genes in the GRPS can function as a genomic biomarker signature to discriminate between respiratory sensitizing chemicals and non-respiratory sensitizing chemicals. Assessment of the predictive performance of GRPS is important in order to establish the reliability of the genomic biomarker signature for identification of respiratory sensitizers. In this study, we used a Support Vector Machine (SVM) algorithm for supervised machine learning. We trained the model to recognize structures and similarities in gene expression data in the identified GRPS genomic biomarker signature, and challenged the model with an independent test set comprising chemicals previously unseen by the model.

Subsequently, we used the model to binary classify the unseen chemical compounds as either respiratory sensitizers or non-respiratory sensitizers. Performing this exercise, we demonstrated the potential of GRPS to achieve accurate predictions. The predictive performance of GRPS was estimated, using ROC AUC analysis and cooper statistics, achieving an area under the ROC curve of 0.97 and sensitivity, specificity and accuracy of 67%, 89% and 84%, respectively. This is the highest reported accuracy for hazard classification of chemicals inducing respiratory sensitization.

To date, we can only speculate on possible explanations to why the GRPS does not reach the same high sensitivity in predictions as the GPS for skin sensitizers [46]. It could partly be due to the smaller number of reference compounds used during assay development of GRPS in comparison to GPS, but another possible explanation could perhaps be found on the molecular level, i.e. that skin sensitizers are more potent regulators of gene expression in MUTZ-3 cells. Irrespectively, the use of whole genome arrays as readout for classifications still makes the GRPS highly flexible. As more samples are analyzed, additional information can easily be implemented into the assay to improve sensitivity, specificity and accuracy and to fine-tune the methodology to reflect the diversity of available chemical compounds.

To further explore the biological effects of sensitizing chemicals on MUTZ-3, an enrichment analysis was performed. In order to achieve sufficient significance in the data, the top 999 genes from p-value filtering were used as input in the Metacore™ software, rather than the top 389 genes of the GRPS. Without doubt, the most highly populated pathways initiated by the respiratory sensitizers were involved in cellular events such as oxidation-reduction processes and respiratory electron transport chain (see table 4). These molecules were among the top genes from the p-value filtering procedure, and not present in the GRPS signature. In this respect, it is important to distinguish between functionality, in this case aiming at describing the biological relevance of the transcripts, and the GRPS prediction profile, aiming at performing accurate classification of independent samples. Several of the molecules involved in the oxidative phosphorylation and ubiquinone metabolism pathways are subunits of protein complexes, and thus spatially and temporally linked. The Backward Elimination procedure applied during feature selection in this study is based on orthogonal selection of variables, thus, features that did not contribute to orthogonal information were removed during this process. Therefore, it is not surprising, but rather expected, that some of the significantly regulated pathways did not contain, or only contained a few transcripts from the GRPS signature as e.g. subunits in a molecular complex will likely have a similar expression pattern. Based on several of the less activated pathways, the biological response in MUTZ-3 to chemical respiratory allergens involves also regulation of innate immune response signalling pathways that ultimately results in cell maturation, leading to enhanced antigen presentation and interaction with other immune cells. Furthermore, novel findings of usage of signalling pathways that has previously been associated with respiratory sensitization to protein allergens will shed light on the biological process leading to sensitization of the respiratory tract in response to chemical allergens. Thus, the GRPS is indeed relevant in an immunologically mechanistic perspective, and provides measurement of transcripts that monitor the biologic events leading to respiratory sensitization.

Further, results from enrichment analysis along with the results presented for the GARD assay, demonstrates that MUTZ-3 is a suitable model for prediction of both skin- and respiratory sensitizers. Despite some similarities in immunobiological mechanisms, important mechanistic differences exist between skin- and respiratory sensitization. Skin sensitization is primary associated with induction of Th1 cells, promoting a cytotoxic CD8+ T-cell response and secretion of IL-2 and interferon (IFN)-γ, while respiratory sensitization generally involve CD4+ Th2 cells and are characterized by high levels of IL-4, IL-10 and IL-13. Although respiratory sensitization to protein antigens are driven by the production of specific IgE antibody, it is still unclear what role the IgE antibody has during the development of respiratory allergy to chemical allergens, and whether there are mechanisms through which respiratory sensitization can be achieved that are independent of IgE antibody production [55,56]. It has been suggested that it may be sufficient with an induced Th2 response, without the need of IgE, to support the development of respiratory sensitization [57]. Although clear differences in T-cell responses, activation of dendritic cells (DCs) is common for both skin- and respiratory sensitization. Consequently, DCs are natural targets for assay development in terms of both skin and respiratory sensitization due to their physiological roles during initiation, modulation and polarization of immune responses in response to xenobiotic compounds. The MUTZ-3 cell line resembles primary dendritic cells (DCs) in terms of expression profile and ability to activate specific T-cell responses [45]. In comparison to primary DCs, MUTZ-3 are easy to grow using standardized protocols and provides a sustainable source of cells, offering an opportunity to scale up the assay to a high-throughput format.

In the context of developing an assay for both skin- and respiratory sensitization, it is important to acknowledge the formal semantics behind the nomenclature. Analogous to others [24,58-60], we use the terminology to indicate the local site of the immunological response and not the route of initial exposure in this study. For example, it has been shown that sensitization of the respiratory tract can arise also after dermal exposure [61-63] to relevant chemicals. In general, a certain chemical compound selectively sensitizes either the skin or the respiratory tract. However, during certain circumstances and in immunologically susceptible individuals, some chemicals have been shown to give rise to both type of sensitization. For example, the chemical triglycidylisocyanurate (TGIC) has been shown to cause both OA and ACD [64].

Finally, the approach of the GARD assay has several advantages, in comparison to other alternative methods. Using our data driven methodology, we were able to circumvent problems associated with the current shortage in knowledge regarding the exact mechanisms by how respiratory sensitizers provoke immunological responses in susceptible individuals. Secondly, the large amount of information obtained by the transcriptome-wide approach provides an additional opportunity to elucidate molecular mechanisms, such as specific signalling or metabolic pathways involved in the process of respiratory sensitization.

The major aim of this study was to develop an in vitro method in accordance with the three Rs principle on reduction, refinement and replacement of animal experiments for prediction of respiratory sensitization. Having trained a model with a set of reference chemicals, we present a tool to determine whether an unknown chemical is likely to behave as a non-respiratory sensitizer or a respiratory sensitizer. In the future, as the gaps in the current knowledge of how chemicals cause sensitization in the respiratory tract continues to be filled in, a consensus similar to the formulation of Adverse Outcome Pathways (AOP) for skin sensitization [67] may be a reality also for testing of respiratory sensitizers. The GRPS will then be an appealing part of an Integrated Testing Strategy (ITS), useful for assessment of DC maturation.

In conclusion, this study presents a predictive biomarker signature for classification of respiratory chemical sensitizers in MUTZ-3 cells that complement the previously described GARD assay for assessment of skin sensitizers. The ability to test for two different endpoints in the same sample provides an attractive and hitherto unique assay for safety assessment of chemicals in an in vitro testing strategy that comply with the three R principle on reduction, refinement and replacement of animal experiments.

### REFERENCES

1. Boverhof D R, Billington R, Gollapudi B B, Hotchkiss J A, Krieger S M, et al. (2008) Respiratory sensitization and allergy: current research approaches and needs. Toxicol Appl Pharmacol 226: 1-13.

2. Banks D E, Tarlo S M (2000) Important issues in occupational asthma. Curr Opin Pulm Med 6: 37-42.

3. Sastre J, Vandenplas O, Park H S (2003) Pathogenesis of occupational asthma. Eur Respir J 22: 364-373.

4. Zammit-Tabona M, Sherkin M, Kijek K, Chan H, Chan-Yeung M (1983) Asthma caused by diphenylmethane diisocyanate in foundry workers. Clinical, bronchial provocation, and immunologic studies. Am Rev Respir Dis 128: 226-230.

5. Bernstein D I, Patterson R, Zeiss C R (1982) Clinical and immunologic evaluation of trimellitic anhydride-and phthalic anhydride-exposed workers using a questionnaire with comparative analysis of enzyme-linked immunosorbent and radioimmunoassay studies. J Allergy Clin Immunol 69: 311-318.

6. Murdoch R D, Pepys J, Hughes E G (1986) IgE antibody responses to platinum group metals: a large scale refinery survey. Br J Ind Med 43: 37-43.

7. Docker A, Wattie J M, Topping M D, Luczynska C M, Newman Taylor A J, et al. (1987) Clinical and immunological investigations of respiratory disease in workers using reactive dyes. Br J Ind Med 44: 534-541.

8. Bourne M S, Flindt M L, Walker J M (1979) Asthma due to industrial use of chloramine. Br Med J 2: 10-12.

9. Tarlo S M, Lemiere C (2014) Occupational asthma. N Engl J Med 370: 640-649.

10. EU (2008) Regulation (EC) no 1272/2008 of the European Parliament and of the Council on classification, labeling and packaging of substances and mixtures, amending and repealing Directives 67/768/EEC and 1999/45/EC, and amending Regulation (EC) no 1907/2006. Annex VI with tables 3.1 and 3.2. Official Journal of the European Union L353/1.

11. Bharadwaj A, Agrawal D K (2004) Immunomodulation in asthma: a distant dream or a close reality? Int Immunopharmacol 4: 495-511.

12. Elias J A, Zhu Z, Chupp G, Homer R J (1999) Airway remodeling in asthma. J Clin Invest 104: 1001-1006.

13. Kopferschmitt-Kubler M C, Ameille J, Popin E, Calastreng-Crinquand A, Vervloet D, et al. (2002) Occupational asthma in France: a 1-yr report of the observatoire National de Asthmes Professionnels project. Eur Respir J 19: 84-89.

14. Munoz X, Cruz M J, Orriols R, Bravo C, Espuga M, et al. (2003) Occupational asthma due to persulfate salts: diagnosis and follow-up. Chest 123: 2124-2129.

15. Orriols R, Costa R, Albanell M, Alberti C, Castejon J, et al. (2006) Reported occupational respiratory diseases in Catalonia. Occup Environ Med 63: 255-260.

16. Verstraelen S, Bloemen K, Nelissen I, Witters H, Schoeters G, et al. (2008) Cell types involved in allergic asthma and their use in in vitro models to assess respiratory sensitization. Toxicol In Vitro 22: 1419-1431.

17. Pauluhn J, Eidmann P, Mohr U (2002) Respiratory hypersensitivity in guinea pigs sensitized to 1,6-hexamethylene diisocyanate (HDI): comparison of results obtained with the monomer and homopolymers of HDI. Toxicology 171: 147-160.

18. Hilton J, Dearman R J, Boylett M S, Fielding I, Basketter D A, et al. (1996) The mouse IgE test for the identification of potential chemical respiratory allergens: considerations of stability and controls. J Appl Toxicol 16: 165-170.

19. Dearman R J, Basketter D A, Kimber I (1992) Variable effects of chemical allergens on serum IgE concentration in mice. Preliminary evaluation of a novel approach to the identification of respiratory sensitizers. J Appl Toxicol 12: 317-323.

20. Arts J H, Droge S C, Spanhaak S, Bloksma N, Penninks A H, et al. (1997) Local lymph node activation and IgE responses in brown Norway and Wistar rats after dermal application of sensitizing and non-sensitizing chemicals. Toxicology 117: 229-234.

21. Warbrick E V, Dearman R J, Kimber I (2002) Induced changes in total serum IgE concentration in the Brown Norway rat: potential for identification of chemical respiratory allergens. J Appl Toxicol 22: 1-11.

22. Dearman R J, Kimber I (2001) Cytokine fingerprinting and hazard assessment of chemical respiratory allergy. J Appl Toxicol 21: 153-163.

23. Dearman R J, Kimber I (1999) Cytokine fingerprinting: characterization of chemical allergens. Methods 19: 56-63.

24. Vanoirbeek J A, Tarkowski M, Vanhooren H M, De Vooght V, Nemery B, et al. (2006) Validation of a mouse model of chemical-induced asthma using trimellitic anhydride, a respiratory sensitizer, and dinitrochlorobenzene, a dermal sensitizer. J Allergy Clin Immunol 117: 1090-1097.

25. Scheerens H, Buckley T L, Davidse E M, Garssen J, Nijkamp F P, et al. (1996) Toluene diisocyanate-induced in vitro tracheal hyperreactivity in the mouse. Am J Respir Crit Care Med 154: 858-865.

26. (EU) D (2010) DIRECTIVE 2010/63/EU OF THE EUROPEAN PARLIAMENT AND OF THE COUNCIL of 22 Sep. 2010 on the protection of animals used for scientific purposes Official Journal of the European Union

27. Verstraelen S, Nelissen I, Hooyberghs J, Witters H, Schoeters G, et al. (2009) Gene profiles of THP-1 macrophages after in vitro exposure to respiratory (non-) sensitizing chemicals: identification of discriminating genetic markers and pathway analysis. Toxicol In Vitro 23: 1151-1162.

28. Verstraelen S, Nelissen I, Hooyberghs J, Witters H, Schoeters G, et al. (2009) Gene profiles of a human bronchial epithelial cell line after in vitro exposure to respiratory (non-)sensitizing chemicals: identification of discriminating genetic markers and pathway analysis. Toxicology 255: 151-159.

29. Verstraelen S, Nelissen I, Hooyberghs J, Witters H, Schoeters G, et al. (2009) Gene profiles of a human alveolar epithelial cell line after in vitro exposure to respiratory (non-)sensitizing chemicals: identification of discriminating genetic markers and pathway analysis. Toxicol Lett 185: 16-22.

30. Huang S, Wiszniewski L, Constant S, Roggen E (2013) Potential of in vitro reconstituted 3D human airway epithelia (MucilAir) to assess respiratory sensitizers. Toxicol In Vitro 27: 1151-1156.

31. Lalko J F, Kimber I, Dearman R J, Gerberick G F, Sarlo K, et al. (2011) Chemical reactivity measurements: potential for characterization of respiratory chemical allergens. Toxicol In Vitro 25: 433-445.

32. Isola D, Kimber I, Sarlo K, Lalko J, Sipes I G (2008) Chemical respiratory allergy and occupational asthma: what are the key areas of uncertainty? J Appl Toxicol 28: 249-253.

33. Kimber I, Agius R, Basketter D A, Corsini E, Cullinan P, et al. (2007) Chemical respiratory allergy: opportunities for hazard identification and characterisation. The report and recommendations of ECVAM workshop 60. Altern Lab Anim 35: 243-265.

34. Mehling A, Eriksson T, Eltze T, Kolle S, Ramirez T, et al. (2012) Non-animal test methods for predicting skin sensitization potentials. Arch Toxicol 86: 1273-1295.

35. Basketter D A, Evans P, Fielder R J, Gerberick G F, Dearman R J, et al. (2002) Local lymph node assay—validation, conduct and use in practice. Food Chem Toxicol 40: 593-598.

36. Ashikaga T, Yoshida Y, Hirota M, Yoneyama K, Itagaki H, et al. (2006) Development of an in vitro skin sensitization test using human cell lines: the human Cell Line Activation Test (h-CLAT). I. Optimization of the h-CLAT protocol. Toxicol In Vitro 20: 767-773.

37. Nukada Y, Ashikaga T, Sakaguchi H, Sono S, Mugita N, et al. (2011) Predictive performance for human skin sensitizing potential of the human cell line activation test (h-CLAT). Contact Dermatitis 65: 343-353.

38. Gerberick G F, Vassallo J D, Bailey R E, Chaney J G, Morrell S W, et al. (2004) Development of a peptide reactivity assay for screening contact allergens. Toxicol Sci 81: 332-343.

39. Natsch A (2010) The Nrf2-Keap1-ARE toxicity pathway as a cellular sensor for skin sensitizers—functional relevance and a hypothesis on innate reactions to skin sensitizers. Toxicol Sci 113: 284-292.

40. Andreas N, Caroline B, Leslie F, Frank G, Kimberly N, et al. (2011) The intra- and inter-laboratory reproducibility and predictivity of the KeratinoSens assay to predict skin sensitizers in vitro: results of a ring-study in five laboratories. Toxicol In Vitro 25: 733-744.

41. Johansson H, Lindstedt M, Albrekt A S, Borrebaeck C A (2011) A genomic biomarker signature can predict skin sensitizers using a cell-based in vitro alternative to animal tests. BMC Genomics 12: 399.

42. Johansson H, Albrekt A S, Borrebaeck C A, Lindstedt M (2013) The GARD assay for assessment of chemical skin sensitizers. Toxicol In Vitro 27: 1163-1169.

43. Santegoets S J, Masterson A J, van der Sluis P C, Lougheed S M, Fluitsma D M, et al. (2006) A CD34(+) human cell line model of myeloid dendritic cell differentiation: evidence for a CD14(+)CD11b(+) Langerhans cell precursor. J Leukoc Biol 80: 1337-1344.

44. Masterson AJ, Sombroek C C, De Gruijl T D, Graus Y M, van der Vliet H J, et al. (2002) MUTZ-3, a human cell line model for the cytokine-induced differentiation of dendritic cells from CD34+ precursors. Blood 100: 701-703.

45. Larsson K, Lindstedt M, Borrebaeck C A (2006) Functional and transcriptional profiling of MUTZ-3, a myeloid cell line acting as a model for dendritic cells. Immunology 117: 156-166.

46. Johansson H, Rydnert F, Kuhnl J, Schepky A, Borrebaeck C, et al. (2014) Genomic allergen rapid detection in-house validation—a proof of concept. Toxicol Sci 139: 362-370.

47. Haneke K E, Tice R R, Carson B L, Margolin B H, Stokes W S (2001) ICCVAM evaluation of the murine local lymph node assay. Data analyses completed by the National Toxicology Program Interagency Center for the Evaluation of Alternative Toxicological Methods. Regul Toxicol Pharmacol 34: 274-286.

48. Vanoirbeek J A, Mandervelt C, Cunningham A R, Hoet P H, Xu H, et al. (2003) Validity of methods to predict the respiratory sensitizing potential of chemicals: A study with a piperidinyl chlorotriazine derivative that caused an outbreak of occupational asthma. Toxicol Sci 76: 338-346.

49. Holsapple M P, Jones D, Kawabata T T, Kimber I, Sarlo K, et al. (2006) Assessing the potential to induce respiratory hypersensitivity. Toxicol Sci 91: 4-13.

50. Kimber I, Basketter D A, Butler M, Gamer A, Garrigue J L, et al. (2003) Classification of contact allergens according to potency: proposals. Food Chem Toxicol 41: 1799-1809.

51. OECD (2002) Guideline 429: Skin Sensitization: Local Lymph Node Assay (2002).

52. Carlsson A, Wingren C, Kristensson M, Rose C, Ferno M, et al. (2011) Molecular serum portraits in patients with primary breast cancer predict the development of distant metastases. Proc Natl Acad Sci USA 108: 14252-14257.

53. Kullback S, Leibler RA (1951) On Information and Sufficiency. Annals of Mathematical Statistics 22: 79-86.

54. Albrekt A S, Johansson H, Borje A, Borrebaeck C, Lindstedt M (2014) Skin sensitizers differentially regulate signaling pathways in MUTZ-3 cells in relation to their individual potency. BMC Pharmacol Toxicol 15: 5.

55. Cartier A, Grammer L, Malo J L, Lagier F, Ghezzo H, et al. (1989) Specific serum antibodies against isocyanates: association with occupational asthma. J Allergy Clin Immunol 84: 507-514.

56. Cullinan P (1998) Occupational asthma, IgE and IgG. Clin Exp Allergy 28: 668-670.

57. Kimber I, Dearman R J, Basketter D A, Boverhof D R (2014) Chemical respiratory allergy: reverse engineering an adverse outcome pathway. Toxicology 318: 32-39.

58. Vanoirbeek J A, Tarkowski M, Ceuppens J L, Verbeken E K, Nemery B, et al. (2004) Respiratory response to toluene diisocyanate depends on prior frequency and concentration of dermal sensitization in mice. Toxicol Sci 80: 310-321.

59. Sailstad D M, Ward M D, Boykin E H, Selgrade M K (2003) A murine model for low molecular weight chemicals: differentiation of respiratory sensitizers (TMA) from contact sensitizers (DNFB). Toxicology 194: 147-161.

60. Pauluhn J (2003) Respiratory hypersensitivity to trimellitic anhydride in Brown Norway rats: analysis of dose-response following topical induction and time course following repeated inhalation challenge. Toxicology 194: 1-17.

61. Karol M H, Hauth B A, Riley E J, Magreni C M (1981) Dermal contact with toluene diisocyanate (TDI) produces respiratory tract hypersensitivity in guinea pigs. Toxicol Appl Pharmacol 58: 221-230.

62. Kimber I, Dearman R J (2002) Chemical respiratory allergy: role of IgE antibody and relevance of route of exposure. Toxicology 181-182: 311-315.

63. Bello D, Herrick C A, Smith T J, Woskie S R, Streicher R P, et al. (2007) Skin exposure to isocyanates: reasons for concern. Environ Health Perspect 115: 328-335.

64. Meuleman L, Goossens A, Linders C, Rochette F, Nemery B (1999) Sensitization to triglycidylisocyanurate (TGIC) with cutaneous and respiratory manifestations. Allergy 54: 752-756.

65. Cary R, Dobson S, Delic J (1999) Concise International Chemical Assessment Document 15 (CICAD): 1,2-DIAMINOETHANE (ETHYLENEDIAMINE). World Health Organization.

66. Commission E (2005) European Union Risk Assessment Report Piperazine. European Chemicals Bureau: European comission. 159 p.

67. OECD (2012) The Adverse Outcome Pathway for Skin Sensitisation Initiated by Covalent Binding to Proteins, Part 1: Scientific Evidence. Environment, Health and Safety Publications, Series on Testing and Assessment No. 168.

68. Hu Z B, Ma W, Zaborski M, MacLeod R, Quentmeier H, et al. (1996) Establishment and characterization of two novel cytokine-responsive acute myeloid and monocytic leukemia cell lines, MUTZ-2 and MUTZ-3. Leukemia 10: 1025-1040.

69. Quentmeier H, Duschl A, Hu Z B, Schnarr B, Zaborski M, et al. (1996) MUTZ-3, a monocytic model cell line for interleukin-4 and lipopolysaccharide studies. Immunology 89: 606-612.

70. Piccolo S R, Sun Y, Campbell J D, Lenburg M E, Bild A H, et al. (2012) A single-sample microarray normalization method to facilitate personalized-medicine workflows. Genomics 100: 337-344.

71. Johnson W E, Li C, Rabinovic A (2007) Adjusting batch effects in microarray expression data using empirical Bayes methods. Biostatistics 8: 118-127.

72. R Development Core Team (2014) R: A Language and Environment for Statistical Computing. R Foundation for Statistical Computing: Vienna, Austria.

73. Gentleman R C, Carey V J, Bates D M, Bolstad B, Dettling M, et al. (2004) Bioconductor: open software development for computational biology and bioinformatics. Genome Biol 5: R80.

74. Leek J T, Johnson W E, Parker H S, Jaffe A E, Storey J D sva: Surrogate Variable Analysis. R package version 3.6.0.

75. Ringner M (2008) What is principal component analysis? Nat Biotechnol 26: 303-304.

76. Benjamini Y H Y (1995) Controlling the False Discovery Rate: A Practical and Powerful Approach to Multiple Testing. Journal of the Royal Statistical Society Series B (Methodological) 57: 12.

77. Lasko T A, Bhagwat J G, Zou K H, Ohno-Machado L (2005) The use of receiver operating characteristic curves in biomedical informatics. J Biomed Inform 38: 404-415.

78. Dimitriadou E, Hornik, K., Leisch, F., Meyer, D., Weingessel, A. (2011) e1071: Misc functions of the Department of statistics (q1071). TU Wien R package version 16 http://CRAN.R-project.org/package=e1071.

79. Cortes C V, V. (1995) Support-Vector Networks. Machine Learning 20: 273-297.

80. Sing T, Sander 0, Beerenwinkel N, Lengauer T (2005) ROCR: visualizing classifier performance in R. Bioinformatics 21: 3940-3941.

81. Cooper J A, 2nd, Saracci R, Cole P (1979) Describing the validity of carcinogen screening tests. Br J Cancer 39: 87-89.

TABLE A
Biomarkers from the GPRS Prediction Signature.

| | | | |
|---|---|---|---|
| | | | Validation |
| | | Affymetrix | call |
| Gene ID | Ensembl Transcript ID | Probe set ID | frequency¹ |

Table A(i) - Core biomarkers

| | | | | |
|---|---|---|---|---|
| 1. | TNFRSF19 | ENST00000403372 | 7968015 | 100 |
| 2. | SNORA74A | NR_002915 | 8108420 | 100 |
| 3. | SPAM1 | ENST00000340011 | 8135835 | 100 |

Table A(ii) - Preferred biomarkers

| | | | | |
|---|---|---|---|---|
| 4. | ETID: ENST00000364621 | ENST00000364621 | 7917972 | 95 |
| 5. | HOMER3 | ENST00000392351 | 8035566 | 95 |
| 6. | CD1C | ENST00000368169 | 7906348 | 90 |
| 7. | IGHD /// IGHM | ENST00000390538 | 7981601 | 90 |
| 8. | SNRPN /// SNORD116-26 | NR_003340 | 7982000 | 90 |
| 9. | ETID: ENST00000364678 | ENST00000364678 | 7934896 | 85 |
| 10. | STRAP | ENST00000025399 | 7954173 | 85 |
| 11. | DIABLO | ENST00000267169 | 7967230 | 85 |
| 12. | ETID: ENST00000411349 | ENST00000411349 | 8151989 | 85 |
| 13. | ETID: ENST00000385497 | ENST00000385497 | 7923037 | 80 |
| 14. | OR51A2 | ENST00000380371 | 7946017 | 80 |
| 15. | MRPL21 | ENST00000362034 | 7949995 | 80 |
| 16. | PPP1R14A | ENST00000301242 | 8036473 | 80 |
| 17. | DEFB127 | ENST00000382388 | 8060314 | 80 |
| 18. | C9orf130 | ENST00000375268 | 8162562 | 80 |
| 19. | PRO2012 | BC019830 | 7924817 | 75 |
| 20. | LOC399898 | AK128188 | 7940116 | 75 |
| 21. | ETID: ENST00000387701 | ENST00000387701 | 7969914 | 75 |
| 22. | WDR68 | ENST00000310827 | 8009164 | 75 |
| 23. | NEU2 | ENST00000233840 | 8049243 | 75 |
| 24. | ETID: ENST00000386677 | ENST00000386677 | 8072575 | 75 |
| 25. | SPARC | ENST00000231061 | 8115327 | 75 |
| 26. | ETID: ENST00000390342 | ENST00000390342 | 8139107 | 75 |
| 27. | CRNN | ENST00000271835 | 7920178 | 70 |
| 28. | MMP12 | ENST00000326227 | 7951297 | 70 |
| 29. | ACVRL1 | ENST00000267008 | 7955562 | 70 |
| 30. | EIF4E2 | ENST00000258416 | 8049180 | 70 |
| 31. | RP11-191L9.1 | ENST00000380990 | 8076819 | 70 |
| 32. | PDCD6 /// AHRR | ENST00000264933 | 8104180 | 70 |
| 33. | ARRDC3 | ENST00000265138 | 8113073 | 70 |
| 34. | VWDE | ENST00000275358 | 8138258 | 70 |
| 35. | ZBTB34 | ENST00000319119 | 8157945 | 70 |
| 36. | ITGB1BP2 | ENST00000373829 | 8168291 | 70 |
| 37. | OR10K2 | ENST00000392265 | 7921356 | 65 |
| 38. | FLJ22596 | AK026249 | 7950442 | 65 |
| 39. | ETID: ENST00000306515 | ENST00000306515 | 8043572 | 65 |
| 40. | ACVR2A | ENST00000404590 | 8045587 | 65 |
| 41. | ETID: ENST00000385690 | ENST00000385690 | 8092312 | 65 |
| 42. | ETID: ENST00000386018 | ENST00000386018 | 8097945 | 65 |
| 43. | C6orf201 | ENST00000360378 | 8116696 | 65 |
| 44. | ETID: ENST00000385583 | ENST00000385583 | 8136932 | 65 |
| 45. | ETID: ENST00000385719 | ENST00000385719 | 8148515 | 65 |
| 46. | GPR20 | ENST00000377741 | 8153269 | 65 |
| 47. | ETID: ENST00000364357 | ENST00000364357 | 8163084 | 65 |
| 48. | ZCCHC13 | ENST00000339534 | 8168420 | 65 |
| 49. | GPR64 | ENST00000356606 | 8171624 | 65 |
| 50. | CD1D | ENST00000368171 | 7906330 | 60 |
| 51. | DUSP12 | ENST00000367943 | 7906810 | 60 |
| 52. | KLHL33 | ENST00000344581 | 7977567 | 60 |
| 53. | PSMB6 | ENST00000270586 | 8003953 | 60 |
| 54. | TMEM95 | ENST00000396580 | 8004364 | 60 |
| 55. | C1QBP | ENST00000225698 | 8011850 | 60 |
| 56. | EMILIN2 | ENST00000254528 | 8019912 | 60 |
| 57. | CD8A | ENST00000352580 | 8053584 | 60 |
| 58. | C20orf152 | ENST00000349339 | 8062237 | 60 |
| 59. | KCNJ4 | ENST00000303592 | 8076072 | 60 |
| 60. | ETID: ENST00000364163 | ENST00000364163 | 8078310 | 60 |
| 61. | FAM19A1 | ENST00000327941 | 8080918 | 60 |
| 62. | ETID: ENST00000384601 | ENST00000384601 | 8081233 | 60 |
| 63. | POLR2H | ENST00000296223 | 8084488 | 60 |
| 64. | AK000420 | AK000420 | 8110706 | 60 |
| 65. | ETID: ENST00000363354 | ENST00000363354 | 8120360 | 60 |
| 66. | APID: 8121483 | — | 8121483 | 60 |
| 67. | EGFL6 | ENST00000361306 | 8166079 | 60 |
| 68. | POU3F4 | ENST00000373200 | 8168567 | 60 |
| 69. | ETID: ENST00000385841 | ENST00000385841 | 7905629 | 55 |
| 70. | OR52A5 | ENST00000307388 | 7946023 | 55 |
| 71. | TIMM8B | ENST00000280354 | 7951679 | 55 |
| 72. | PEBP1 | ENST00000261313 | 7959070 | 55 |
| 73. | OR4F6 | ENST00000328882 | 7986530 | 55 |
| 74. | CDH15 | ENST00000289746 | 7997880 | 55 |
| 75. | TMEM199 | ENST00000292114 | 8005857 | 55 |
| 76. | ABI3 | ENST00000225941 | 8008185 | 55 |
| 77. | FLJ42842 | AK124832 | 8008540 | 55 |
| 78. | MC4R | ENST00000299766 | 8023593 | 55 |
| 79. | ETID: ENST00000410673 | ENST00000410673 | 8045931 | 55 |
| 80. | ISM1 | ENST00000262487 | 8061013 | 55 |
| 81. | LOC440957 | ENST00000307106 | 8080416 | 55 |
| 82. | KLB | ENST00000257408 | 8094679 | 55 |
| 83. | GM2A | ENST00000357164 | 8109344 | 55 |
| 84. | ANXA6 | ENST00000354546 | 8115234 | 55 |
| 85. | TAS2R40 | ENST00000408947 | 8136846 | 55 |
| 86. | APID: 8142880 | — | 8142880 | 55 |
| 87. | RARRES2 | ENST00000223271 | 8143772 | 55 |
| 88. | SH2D4A | ENST00000265807 | 8144880 | 55 |
| 89. | PLP1 | ENST00000361621 | 8169061 | 55 |
| 90. | ATP1A2 | ENST00000392233 | 7906501 | 50 |
| 91. | ETID: ENST00000386800 | ENST00000386800 | 7932610 | 50 |
| 92. | MAT1A | ENST00000372206 | 7934755 | 50 |
| 93. | TSGA10IP | ENST00000312452 | 7941469 | 50 |
| 94. | PRDM7 | ENST00000325921 | 8003571 | 50 |
| 95. | ETID: ENST00000390847 | ENST00000390847 | 8015739 | 50 |
| 96. | ETID: ENST00000255183 | ENST00000255183 | 8066444 | 50 |
| 97. | MRPL39 | ENST00000307301 | 8069620 | 50 |
| 98. | ETID: ENST00000386327 | ENST00000386327 | 8074884 | 50 |
| 99. | TIPARP | ENST00000295924 | 8083569 | 50 |
| 100. | HES1 | ENST00000232424 | 8084880 | 50 |
| 101. | ETID: ENST00000363502 | ENST00000363502 | 8089727 | 50 |
| 102. | PRDM9 | ENST00000253473 | 8104634 | 50 |
| 103. | ETID: ENST00000390917 | ENST00000390917 | 8137433 | 50 |
| 104. | KIAA1688 | ENST00000377307 | 8153876 | 50 |
| 105. | ETID: ENST00000391219 | ENST00000391219 | 8156759 | 50 |
| 106. | ETID: ENST00000387973 | ENST00000387973 | 8160782 | 50 |
| 107. | LOC100129534 | — | 7911718 | 45 |
| 108. | SLC2A1 | ENST00000397019 | 7915472 | 45 |
| 109. | AF116714 | AF116714 | 7935359 | 45 |
| 110. | EPS8L2 | ENST00000318562 | 7937443 | 45 |
| 111. | MGC3196 | ENST00000307366 | 7948836 | 45 |
| 112. | 7952733 | — | 7952733 | 45 |
| 113. | ETID: ENST00000384391 | ENST00000384391 | 7990031 | 45 |
| 114. | EME2 | ENST00000307394 | 7992379 | 45 |
| 115. | NETO1 | ENST00000299430 | 8023828 | 45 |
| 116. | NPHS1 | ENST00000353632 | 8036176 | 45 |
| 117. | ETID: ENST00000384109 | ENST00000384109 | 8047215 | 45 |
| 118. | ETID: ENST00000364143 | ENST00000364143 | 8059799 | 45 |
| 119. | ISX | ENST00000404699 | 8072636 | 45 |
| 120. | IL17RB | ENST00000288167 | 8080562 | 45 |
| 121. | PCOLCE2 | ENST00000295992 | 8091243 | 45 |
| 122. | LRIT3 | ENST00000409621 | 8096839 | 45 |
| 123. | ETID: ENST00000330110 | ENST00000330110 | 8104615 | 45 |
| 124. | ZNF354C | ENST00000315475 | 8110491 | 45 |
| 125. | ETID: ENST00000386444 | ENST00000386444 | 8162927 | 45 |
| 126. | OR2G3 | ENST00000320002 | 7911209 | 40 |
| 127. | GLUL | ENST00000331872 | 7922689 | 40 |
| 128. | CCKBR | ENST00000334619 | 7938090 | 40 |
| 129. | OR1S2 | ENST00000302592 | 7948312 | 40 |
| 130. | DCUN1D5 | ENST00000260247 | 7951325 | 40 |
| 131. | ETID: ENST00000388291 | ENST00000388291 | 7951420 | 40 |
| 132. | EMG1 | ENST00000261406 | 7953594 | 40 |
| 133. | PTHLH | ENST00000395868 | 7962000 | 40 |
| 134. | PTGES3 | ENST00000262033 | 7964250 | 40 |
| 135. | CIDEB | ENST00000258807 | 7978272 | 40 |
| 136. | ETID: ENST00000383863 | ENST00000383863 | 7985918 | 40 |
| 137. | ATP10A | ENST00000356865 | 7986789 | 40 |
| 138. | MYO5C | ENST00000261839 | 7988876 | 40 |
| 139. | ETID: ENST00000380078 | ENST00000380078 | 7989951 | 40 |
| 140. | PLA2G10 | ENST00000261659 | 7999588 | 40 |
| 141. | HSPE1 | ENST00000409729 | 8047223 | 40 |
| 142. | ETID: ENST00000388324 | ENST00000388324 | 8096249 | 40 |
| 143. | MYO6 | ENST00000369977 | 8120783 | 40 |
| 144. | C7orf30 | ENST00000287543 | 8131860 | 40 |
| 145. | ETID: ENST00000340779 | ENST00000340779 | 8139828 | 40 |
| 146. | LOC441245 | AK090474 | 8139887 | 40 |
| 147. | CRIM2 | ENST00000297801 | 8142821 | 40 |
| 148. | XKR4 | ENST00000327381 | 8146475 | 40 |
| 149. | FAM110B | ENST00000361488 | 8146533 | 40 |
| 150. | PEBP4 | ENST00000256404 | 8149725 | 40 |
| 151. | LOC644714 | BC047037 | 8161943 | 40 |
| 152. | PAPPAS | AY623011 /// AY623012 | 8163672 | 40 |
| 153. | BEX4 | ENST00000372691 | 8169009 | 40 |
| 154. | HMGB4 | ENST00000323936 | 7899905 | 35 |
| 155. | ETID: BC028413 /// BC128516 | BC028413 /// BC128516 | 7911676 | 35 |
| 156. | ETID: ENST00000363919 | ENST00000363919 | 7928750 | 35 |
| 157. | ETID: ENST00000335621 | ENST00000335621 | 7958942 | 35 |
| 158. | SOX1 | ENST00000330949 | 7970146 | 35 |
| 159. | CTSG | ENST00000216336 | 7978351 | 35 |
| 160. | ETID: ENST00000362344 | ENST00000362344 | 7982100 | 35 |
| 161. | FLJ37464 | ENST00000398354 | 7996377 | 35 |
| 162. | RAX | ENST00000334889 | 8023549 | 35 |
| 163. | IL29 | ENST00000333625 | 8028613 | 35 |
| 164. | CEACAM20 | ENST00000316962 | 8037482 | 35 |
| 165. | ETID: ETID: ENST00000365557 | ENST00000365557 | 8044684 | 35 |
| 166. | SEC14L3 | ENST00000403066 | 8075375 | 35 |
| 167. | C3orf52 | ENST00000264848 | 8081645 | 35 |
| 168. | FETUB | ENST00000265029 | 8084657 | 35 |
| 169. | PIGY | ENST00000273968 | 8101718 | 35 |
| 170. | CDH12 | ENST00000284308 | 8111234 | 35 |
| 171. | LGSN | ENST00000370657 | 8127380 | 35 |
| 172. | ETID: ENST00000391031 | ENST00000391031 | 8129067 | 35 |
| 173. | HGC6.3 | AB016902 | 8130824 | 35 |
| 174. | tcag7.873 | NM_001126493 | 8138797 | 35 |
| 175. | T1560 | ENST00000379496 | 8146527 | 35 |
| 176. | EXOSC4 | ENST00000316052 | 8148710 | 35 |
| 177. | TRAM1 | ENST00000262213 | 8151281 | 35 |
| 178. | APID: 8159371 | — | 8159371 | 35 |
| 179. | OR13C2 | ENST00000318797 | 8162936 | 35 |
| 180. | PLS3 | ENST00000289290 | 8169473 | 35 |
| 181. | TMEM53 | ENST00000372244 | 7915578 | 30 |
| 182. | CD1B | ENST00000368168 | 7921346 | 30 |
| 183. | SORCS3 | ENST00000393176 | 7930341 | 30 |
| 184. | OR52E8 | ENST00000329322 | 7946111 | 30 |
| 185. | FAM160A2 | ENST00000265978 | 7946128 | 30 |
| 186. | LOC649946 | BC017930 | 7952126 | 30 |
| 187. | FAM158A | ENST00000216799 | 7978114 | 30 |
| 188. | APID: 7986637 | — | 7986637 | 30 |
| 189. | MYO1E | ENST00000288235 | 7989277 | 30 |
| 190. | NUPR1 | ENST00000395641 | 8000574 | 30 |
| 191. | APID: 8005433 | — | 8005433 | 30 |
| 192. | SIGLEC15 | ENST00000389474 | 8021091 | 30 |
| 193. | 2-Mar | ENST00000393944 | 8025421 | 30 |
| 194. | LOC100131554 | — | 8041886 | 30 |
| 195. | GGTLC1 | ENST00000335694 | 8065427 | 30 |
| 196. | PSMA7 | ENST00000395567 | 8067382 | 30 |
| 197. | SLC25A18 | ENST00000399813 | 8071107 | 30 |
| 198. | C3orf14 | ENST00000232519 | 8080847 | 30 |
| 199. | CDX1 | ENST00000377812 | 8109226 | 30 |
| 200. | ETID: ENST00000386433 | ENST00000386433 | 8121249 | 30 |
| 201. | RRAGD | ENST00000359203 | 8128123 | 30 |
| 202. | SDK1 | ENST00000389531 | 8131205 | 30 |
| 203. | LOC168474 | NR_002789 | 8139826 | 30 |
| 204. | ETID: ENST00000384125 | ENST00000384125 | 8146120 | 30 |
| 205. | TRHR | ENST00000311762 | 8147877 | 30 |
| 206. | IL11RA | ENST00000378817 | 8154934 | 30 |
| 207. | MGC21881 /// LOC554249 | ENST00000377616 | 8155393 | 30 |
| 208. | ZNF483 | ENST00000358151 | 8157193 | 30 |
| 209. | C9orf169 | ENST00000400709 | 8159624 | 30 |
| 210. | MGC21881 /// LOC554249 | ENST00000377616 | 8161451 | 30 |
| 211. | ETID: ENST00000364507 | ENST00000364507 | 8168161 | 30 |
| 212. | ETID: ENST00000387003 | ENST00000387003 | 7914137 | 25 |
| 213. | ETID: ENST00000388083 | ENST00000388083 | 7929614 | 25 |
| 214. | ETID: ENST00000365084 | ENST00000365084 | 7934568 | 25 |
| 215. | FRG2 /// FRG2B /// FRG2C | ENST00000368515 | 7937251 | 25 |
| 216. | C14orf53 | ENST00000389594 | 7975154 | 25 |
| 217. | ODF3L1 | ENST00000332145 | 7985025 | 25 |
| 218. | FAM18A | ENST00000299866 | 7999412 | 25 |
| 219. | PRTN3 | ENST00000234347 | 8024048 | 25 |
| 220. | CFD | ENST00000327726 | 8024062 | 25 |
| 221. | TMED1 | ENST00000214869 | 8034101 | 25 |
| 222. | ETID: ENST00000387150 | ENST00000387150 | 8035937 | 25 |
| 223. | HSD17B14 | ENST00000263278 | 8038213 | 25 |
| 224. | BOK | ENST00000318407 | 8049876 | 25 |
| 225. | ETID: ENST00000365609 | ENST00000365609 | 8050801 | 25 |
| 226. | SNRPB | ENST00000381342 | 8064502 | 25 |
| 227. | EPHA6 | ENST00000338994 | 8081138 | 25 |
| 228. | SCARNA22 | NR_003004 | 8093576 | 25 |
| 229. | FLJ35424 | ENST00000404649 | 8093821 | 25 |
| 230. | ETID: ENST00000387555 | ENST00000387555 | 8104723 | 25 |
| 231. | ETID: ENST00000388664 | ENST00000388664 | 8107115 | 25 |
| 232. | ETID: ENST00000363365 | ENST00000363365 | 8108566 | 25 |
| 233. | ETID: ENST00000362861 | ENST00000362861 | 8111358 | 25 |
| 234. | ETID: ENST00000363181 | ENST00000363181 | 8114581 | 25 |
| 235. | GRM6 | ENST00000319065 | 8116253 | 25 |
| 236. | LOC646093 | — | 8116400 | 25 |
| 237. | HIST1H1E | ENST00000304218 | 8117377 | 25 |
| 238. | TIAM2 | ENST00000367174 | 8122933 | 25 |
| 239. | ETID: ENST00000363074 | ENST00000363074 | 8128712 | 25 |
| 240. | ETID: ENST00000385777 | ENST00000385777 | 8148331 | 25 |
| 241. | MTUS1 | ENST00000400046 | 8149500 | 25 |
| 242. | MUC21 | ENST00000383351 | 8177931 | 25 |
| 243. | WDR8 | ENST00000378322 | 7911839 | 20 |
| 244. | LOC100131195 | AK097743 | 7933190 | 20 |
| 245. | OR4D10 | ENST00000378245 | 7940182 | 20 |
| 246. | C12orf63 | ENST00000342887 | 7957688 | 20 |
| 247. | ELA1 | ENST00000293636 | 7963304 | 20 |
| 248. | DNAJC14 /// CIP29 | ENST00000317269 | 7963935 | 20 |
| 249. | FLJ40176 | ENST00000322527 | 7972670 | 20 |
| 250. | ETID: ENST00000410207 | ENST00000410207 | 7985308 | 20 |
| 251. | PSME3 | ENST00000293362 | 8007397 | 20 |
| 252. | ETID: ENST00000405656 | ENST00000405656 | 8009515 | 20 |
| 253. | HN1 | ENST00000356033 | 8018305 | 20 |
| 254. | ETID: ENST00000335523 | ENST00000335523 | 8027385 | 20 |
| 255. | CYP2A7 /// CYP2A7P1 | ENST00000301146 | 8036981 | 20 |
| 256. | ATXN10 | ENST00000252934 | 8073799 | 20 |
| 257. | ZMAT5 | ENST00000397779 | 8075276 | 20 |
| 258. | ETID: ENST00000362493 | ENST00000362493 | 8084215 | 20 |
| 259. | FHIT | ENST00000341848 | 8088458 | 20 |
| 260. | FRG2 /// FRG2B /// FRG2C | ENST00000368515 | 8104124 | 20 |
| 261. | SNX18 | ENST00000381410 | 8105328 | 20 |
| 262. | ETID: ENST00000362433 | ENST00000362433 | 8128445 | 20 |
| 263. | DTX2 | ENST00000307569 | 8133736 | 20 |
| 264. | ASB4 | ENST00000325885 | 8134376 | 20 |
| 265. | ETID: ENST00000365242 | ENST00000365242 | 8147445 | 20 |
| 266. | ETID: ENST00000364204 | ENST00000364204 | 8156450 | 20 |
| 267. | COL5A1 | ENST00000355306 | 8159142 | 20 |
| 268. | LCAP | ENST00000357566 | 8170786 | 20 |
| 269. | APOO | ENST00000379226 | 8171823 | 20 |
| 270. | PTPRU | ENST00000373779 | 7899562 | 15 |
| 271. | IL28RA | ENST00000327535 | 7913776 | 15 |
| 272. | NEUROG3 | ENST00000242462 | 7934083 | 15 |
| 273. | VAX1 | ENST00000277905 | 7936552 | 15 |
| 274. | LOC440131 | ENST00000400540 | 7968323 | 15 |
| 275. | C13orf31 | ENST00000325686 | 7968883 | 15 |
| 276. | ADAMTS7 | ENST00000388820 | 7990736 | 15 |
| 277. | SMTNL2 | ENST00000338859 | 8003892 | 15 |
| 278. | LOC284112 | AK098506 | 8012004 | 15 |
| 279. | ETV2 | ENST00000402764 | 8027920 | 15 |
| 280. | FUT2 | ENST00000391876 | 8030094 | 15 |
| 281. | C2orf39 | ENST00000288710 | 8040672 | 15 |
| 282. | LOC200383 /// DNAH6 | ENST00000237449 | 8043071 | 15 |
| 283. | ETID: ENST00000385676 | ENST00000385676 | 8055204 | 15 |
| 284. | CCDC108 | ENST00000341552 | 8059028 | 15 |
| 285. | APID: 8065011 | — | 8065011 | 15 |
| 286. | C22orf27 | BC042980 | 8072400 | 15 |
| 287. | ETID: ENST00000364444 | ENST00000364444 | 8103041 | 15 |
| 288. | PDLIM3 | ENST00000284767 | 8104022 | 15 |
| 289. | ETID: ENST00000330110 | ENST00000330110 | 8104613 | 15 |
| 290. | ETID: ENST00000384539 | ENST00000384539 | 8107125 | 15 |
| 291. | ETID: ENST00000390214 | ENST00000390214 | 8130372 | 15 |
| 292. | MGC72080 | BC029615 | 8141169 | 15 |
| 293. | C9orf128 | ENST00000377984 | 8161154 | 15 |
| 294. | RGAG4 | NM_001024455 | 8173503 | 15 |
| 295. | PIP5K1A | ENST00000409426 | 7905365 | 10 |
| 296. | GPR161 | ENST00000367838 | 7922108 | 10 |
| 297. | ETID: ENST00000385353 | ENST00000385353 | 7925434 | 10 |
| 298. | OR56A3 | ENST00000329564 | 7938066 | 10 |
| 299. | OR5A2 | ENST00000302040 | 7948377 | 10 |
| 300. | WNT11 | ENST00000322563 | 7950534 | 10 |
| 301. | APID: 7960259 | — | 7960259 | 10 |
| 302. | RAB37 | ENST00000340415 | 8009666 | 10 |
| 303. | LAIR1 | ENST00000391742 | 8039257 | 10 |
| 304. | ETID: ENST00000388385 | ENST00000388385 | 8041420 | 10 |
| 305. | CHAC2 | ENST00000295304 | 8041961 | 10 |
| 306. | ETID: ENST00000387574 | ENST00000387574 | 8062337 | 10 |
| 307. | ETID: ENST00000387884 | ENST00000387884 | 8062962 | 10 |
| 308. | BCL2L1 | ENST00000376062 | 8065569 | 10 |
| 309. | KDELR3 | ENST00000409006 | 8073015 | 10 |
| 310. | TMEM108 | ENST00000321871 | 8082767 | 10 |
| 311. | SPATA16 | ENST00000351008 | 8092187 | 10 |
| 312. | BTC | ENST00000395743 | 8101002 | 10 |
| 313. | SUPT3H | ENST00000371460 | 8126710 | 10 |
| 314. | EIF4B | ENST00000262056 | 8135268 | 10 |
| 315. | CHMP4C | ENST00000297265 | 8147057 | 10 |
| 316. | H2BFM | ENST00000243297 | 8169080 | 10 |
| 317. | APID: 8180392 | — | 8180392 | 10 |
| 318. | NR5A2 | ENST00000367362 | 7908597 | 5 |
| 319. | TRIM49 | ENST00000332682 | 7939884 | 5 |
| 320. | MS4A6A | ENST00000323961 | 7948455 | 5 |
| 321. | C11orf10 | ENST00000257262 | 7948606 | 5 |
| 322. | HSPC152 | ENST00000308774 | 7949075 | 5 |
| 323. | RASAL1 | ENST00000261729 | 7966542 | 5 |
| 324. | ETID: ENST00000387531 | ENST00000387531 | 7975694 | 5 |
| 325. | PLDN | ENST00000220531 | 7983502 | 5 |
| 326. | PER1 | ENST00000354903 | 8012349 | 5 |
| 327. | ALS2CR12 | ENST00000286190 | 8058203 | 5 |
| 328. | C20orf142 | ENST00000396825 | 8066407 | 5 |
| 329. | ETID: ENST00000386848 | ENST00000386848 | 8073680 | 5 |
| 330. | LOC100129113 | AK094477 | 8074307 | 5 |
| 331. | CERK | ENST00000216264 | 8076792 | 5 |
| 332. | ETID: ENST00000385783 | ENST00000385783 | 8083937 | 5 |
| 333. | PROS1 | ENST00000407433 | 8089015 | 5 |
| 334. | PCDHGA | ENST00000378105 | 8108757 | 5 |
| 335. | MUC3B /// MUC3A | ENST00000332750 | 8135015 | 5 |
| 336. | ETID: ENST00000365355 | ENST00000365355 | 8142534 | 5 |
| 337. | APID: 8156969 | — | 8156969 | 5 |
| 338. | ETID: ENST00000358047 | ENST00000410626 | 8163013 | 5 |
| 339. | FAM47C | ENST00000358047 | 8166703 | 5 |
| 340. | NXF4 | ENST00000360035 | 8168940 | 5 |
| 341. | PIWIL4 | ENST00000299001 | 7943240 | 0 |
| 342. | ETID: ENST00000384727 | ENST00000384727 | 7968732 | 0 |
| 343. | ALDH6A1 | ENST00000350259 | 7980098 | 0 |
| 344. | TNAENA64 | ENST00000324979 | 8151747 | 0 |
| 345. | ETID: ENST00000364816 | ENST00000364816 | 8168079 | 0 |

Table A(iii) - Optional biomarkers

| | | | | |
|---|---|---|---|---|
| 346. | C11orf73 | ENST00000278483 | 7942932 | 100 |
| 347. | OR5B21 | ENST00000278483 | 7948330 | 100 |
| 348. | NOX5 /// SPESP1 | ENST00000395421 | 7984488 | 100 |
| 349. | AMICA1 | ENST00000356289 | 7952022 | 95 |
| 350. | ETID: ENST00000387422 | ENST00000387422 | 8159963 | 90 |
| 351. | SERPINB1 | ENST00000380739 | 8123598 | 85 |
| 352. | ETID: ENST00000387396 | ENST00000387396 | 8065752 | 80 |
| 353. | CD1A | ENST00000289429 | 7906339 | 75 |
| 354. | RAB9A | ENST00000243325 | 8166098 | 75 |
| 355. | C10orf90 | ENST00000356858 | 7936996 | 70 |
| 356. | LPXN | ENST00000263845 | 7948332 | 65 |
| 357. | GGTLC2 | ENST00000215938 | 8071662 | 65 |
| 358. | ETID: ENST00000384680 | ENST00000384680 | 8051862 | 60 |
| 359. | PNPLA4 | ENST00000381042 | 8171229 | 60 |
| 360. | CAMK1D | ENST00000378845 | 7926223 | 55 |
| 361. | ETID: ENST00000410754 | ENST00000410754 | 8120979 | 55 |
| 362. | CDC123 | ENST00000281141 | 7926207 | 50 |
| 363. | WDFY1 | ENST00000233055 | 8059361 | 50 |
| 364. | hCG_1749005 | — | 8167640 | 50 |
| 365. | CD48 | ENST00000368046 | 7921667 | 45 |
| 366. | MED19 | ENST00000337672 | 7948293 | 45 |
| 367. | DRD5 | ENST00000304374 | 8053725 | 45 |
| 368. | APID: 7967586 | — | 7967586 | 40 |
| 369. | VAPA | ENST00000340541 | 8020129 | 40 |
| 370. | FAM71F1 | ENST00000315184 | 8135945 | 40 |
| 371. | APID: 8141421 | — | 8141421 | 35 |
| 372. | HCCS | ENST00000321143 | 8165995 | 35 |
| 373. | CNR2 | ENST00000374472 | 7913705 | 25 |
| 374. | OIT3 | ENST00000334011 | 7928330 | 25 |
| 375. | BMP2K | ENST00000335016 | 8096004 | 25 |
| 376. | ZNF366 | ENST00000318442 | 8112584 | 25 |
| 377. | SYT17 | ENST00000396244 | 7993624 | 20 |
| 378. | CALM12 | ENST00000272298 | 8052010 | 20 |
| 379. | XK | ENST00000378616 | 8166723 | 20 |
| 380. | ART4 | ENST00000228936 | 7961507 | 15 |
| 381. | ETID: ENST00000332418 | ENST00000332418 | 7997907 | 15 |
| 382. | ZFP36L2 | ENST00000282388 | 8051814 | 15 |
| 383. | GSTA3 | ENST00000370968 | 8127087 | 15 |
| 384. | COL21A1 | ENST00000370817 | 8127201 | 15 |
| 385. | ETID: ENST00000332418 | ENST00000332418 | 8170322 | 15 |
| 386. | FUCA1 | ENST00000374479 | 7913694 | 5 |
| 387. | ETID: ENST00000386628 | ENST00000386628 | 7925821 | 5 |
| 388. | AZU1 | ENST00000334630 | 8024038 | 5 |
| 389. | IL7R | ENST00000303115 | 8104901 | 5 |

The table shows predictor genes in GRPS, identified by one-way ANOVA p-value filtering and Backward elimination. When possible, the Ensembl transcript ID was used as gene identifier. The Affymetrix Probe Set ID for the Human ST 1.0 Array are provided.

¹Validation call frequency (%) describes the occurrence of each predictor transcript among the 20 biomarker signatures obtained by cross validation.

TABLE B
GRPS predictor gene expression trends upon sensitizer exposure

| Probe ID | Up/down |
|---|---|
| 7899905 | down |
| 7905365 | up |
| 7905629 | up |
| 7906330 | up |
| 7906339 | up |
| 7906348 | up |
| 7906501 | down |
| 7906810 | up |
| 7908597 | up |
| 7911209 | down |
| 7911676 | up |
| 7911718 | up |
| 7911839 | up |
| 7913694 | up |
| 7913705 | up |
| 7913776 | down |
| 7914137 | up |
| 7915472 | up |
| 7915578 | up |
| 7917972 | down |
| 7920178 | down |
| 7921346 | up |
| 7921356 | up |
| 7921667 | up |
| 7922108 | down |
| 7922689 | down |
| 7923037 | up |
| 7924817 | down |
| 7925434 | down |
| 7925821 | up |
| 7926207 | up |
| 7926223 | up |
| 7928330 | up |
| 7928750 | up |
| 7929614 | up |
| 7930341 | down |
| 7932610 | up |
| 7933190 | up |
| 7934083 | down |
| 7934568 | up |
| 7934755 | up |
| 7934896 | up |
| 7935359 | down |
| 7936552 | down |
| 7936996 | up |
| 7937251 | up |
| 7937443 | up |
| 7938066 | up |
| 7938090 | down |
| 7939884 | up |
| 7940116 | down |
| 7940182 | up |
| 7941469 | down |
| 7942932 | up |
| 7943240 | down |
| 7946017 | up |
| 7946023 | down |
| 7946111 | down |
| 7946128 | down |
| 7948293 | up |
| 7948312 | down |
| 7948330 | up |
| 7948332 | up |
| 7948377 | up |
| 7948455 | up |
| 7948606 | up |
| 7948836 | up |
| 7949075 | up |
| 7949995 | up |
| 7950442 | down |
| 7950534 | up |
| 7951297 | up |
| 7951325 | up |
| 7951420 | up |
| 7951679 | up |
| 7952022 | up |
| 7952126 | up |
| 7952733 | up |
| 7953594 | up |
| 7954173 | up |
| 7955562 | up |
| 7957688 | up |
| 7958942 | up |
| 7959070 | up |
| 7960259 | up |
| 7961507 | up |
| 7962000 | down |
| 7963304 | down |
| 7963935 | up |
| 7964250 | up |
| 7966542 | up |
| 7967230 | up |
| 7967586 | down |
| 7968015 | down |
| 7968323 | up |
| 7968732 | up |
| 7968883 | up |
| 7969914 | up |
| 7970146 | down |
| 7972670 | up |
| 7975154 | up |
| 7975694 | down |
| 7977567 | down |
| 7978114 | up |
| 7978272 | up |
| 7978351 | down |
| 7980098 | down |
| 7981601 | down |
| 7982000 | down |
| 7982100 | up |
| 7983502 | down |
| 7984488 | up |
| 7985025 | up |
| 7985308 | up |
| 7985918 | down |
| 7986530 | down |
| 7986637 | down |
| 7986789 | up |
| 7988876 | up |
| 7989277 | up |
| 7989951 | down |
| 7990031 | down |
| 7990736 | down |
| 7992379 | down |
| 7993624 | down |
| 7996377 | up |
| 7997880 | down |
| 7997907 | up |
| 7999412 | up |
| 7999588 | up |
| 8000574 | down |
| 8003571 | up |
| 8003892 | up |
| 8003953 | up |
| 8004364 | up |
| 8005433 | up |
| 8005857 | up |
| 8007397 | up |
| 8008185 | up |
| 8008540 | down |
| 8009164 | up |
| 8009515 | up |
| 8009666 | down |
| 8011850 | up |
| 8012004 | up |
| 8012349 | down |
| 8015739 | down |
| 8018305 | up |
| 8019912 | up |
| 8020129 | up |
| 8021091 | down |
| 8023549 | down |
| 8023593 | down |
| 8023828 | up |
| 8024038 | down |
| 8024048 | down |
| 8024062 | down |
| 8025421 | down |
| 8027385 | down |
| 8027920 | down |
| 8028613 | up |
| 8030094 | up |
| 8034101 | up |
| 8035566 | down |
| 8035937 | down |
| 8036176 | down |
| 8036473 | down |
| 8036981 | down |
| 8037482 | down |
| 8038213 | down |
| 8039257 | down |
| 8040672 | up |
| 8041420 | down |
| 8041886 | down |
| 8041961 | up |
| 8043071 | up |
| 8043572 | up |
| 8044684 | up |
| 8045587 | up |
| 8045931 | down |
| 8047215 | down |
| 8047223 | up |
| 8049180 | up |
| 8049243 | down |
| 8049876 | up |
| 8050801 | down |
| 8051814 | down |
| 8051862 | up |
| 8052010 | up |
| 8053584 | down |
| 8053725 | up |
| 8055204 | down |
| 8058203 | up |
| 8059028 | down |
| 8059361 | down |
| 8059799 | up |
| 8060314 | up |
| 8061013 | up |
| 8062237 | up |
| 8062337 | down |
| 8062962 | down |
| 8064502 | up |
| 8065011 | up |
| 8065427 | up |
| 8065569 | down |
| 8065752 | down |
| 8066407 | up |
| 8066444 | down |
| 8067382 | up |
| 8069620 | up |
| 8071107 | up |
| 8071662 | down |
| 8072400 | down |
| 8072575 | down |
| 8072636 | up |
| 8073015 | down |
| 8073680 | up |
| 8073799 | up |
| 8074307 | down |
| 8074884 | up |
| 8075276 | up |
| 8075375 | down |
| 8076072 | down |
| 8076792 | down |
| 8076819 | up |
| 8078310 | up |
| 8080416 | up |
| 8080562 | up |
| 8080847 | down |
| 8080918 | down |
| 8081138 | up |
| 8081233 | down |
| 8081645 | up |
| 8082767 | up |
| 8083569 | up |
| 8083937 | up |
| 8084215 | up |
| 8084488 | up |
| 8084657 | up |
| 8084880 | down |
| 8088458 | down |
| 8089015 | down |
| 8089727 | down |
| 8091243 | down |
| 8092187 | up |
| 8092312 | down |
| 8093576 | up |
| 8093821 | down |
| 8094679 | up |
| 8096004 | down |
| 8096249 | up |
| 8096839 | down |
| 8097945 | down |
| 8101002 | down |
| 8101718 | up |
| 8103041 | down |
| 8104022 | down |
| 8104124 | up |
| 8104180 | up |
| 8104613 | up |
| 8104615 | up |
| 8104634 | up |
| 8104723 | up |
| 8104901 | down |
| 8105328 | down |
| 8107115 | down |
| 8107125 | down |
| 8108420 | up |
| 8108566 | down |
| 8108757 | up |
| 8109226 | down |
| 8109344 | down |
| 8110491 | up |
| 8110706 | up |
| 8111234 | down |
| 8111358 | up |
| 8112584 | up |
| 8113073 | down |
| 8114581 | down |
| 8115234 | down |
| 8115327 | down |
| 8116253 | up |
| 8116400 | up |
| 8116696 | down |
| 8117377 | up |
| 8120360 | up |
| 8120783 | down |
| 8120979 | up |
| 8121249 | down |
| 8121483 | up |
| 8122933 | down |
| 8123598 | down |
| 8127087 | down |
| 8127201 | up |
| 8127380 | up |
| 8128123 | down |
| 8128445 | up |
| 8128712 | down |
| 8129067 | down |
| 8130372 | down |
| 8130824 | down |
| 8131205 | down |
| 8131860 | up |
| 8133736 | up |
| 8134376 | up |
| 8135015 | up |
| 8135268 | down |
| 8135835 | down |
| 8135945 | up |
| 8136846 | up |
| 8136932 | down |
| 8137433 | up |
| 8138258 | up |
| 8138797 | up |
| 8139107 | down |
| 8139826 | down |
| 8139828 | down |
| 8139887 | up |
| 8141169 | up |
| 8141421 | down |
| 8142534 | down |
| 8142821 | down |
| 8142880 | down |
| 8143772 | down |
| 8144880 | down |
| 8146120 | up |
| 8146475 | down |
| 8146527 | down |
| 8146533 | up |
| 8147057 | up |
| 8147445 | up |
| 8147877 | up |
| 8148331 | up |
| 8148515 | up |
| 8148710 | up |
| 8149500 | down |
| 8149725 | up |
| 8151281 | down |
| 8151747 | down |
| 8151989 | up |
| 8153269 | up |
| 8153876 | down |
| 8154934 | down |
| 8155393 | up |
| 8156450 | down |
| 8156759 | down |
| 8156969 | up |
| 8157193 | down |
| 8157945 | down |
| 8159142 | down |
| 8159371 | up |
| 8159624 | up |
| 8159963 | down |
| 8160782 | down |
| 8161154 | down |
| 8161451 | up |
| 8161943 | up |
| 8162562 | down |
| 8162927 | down |
| 8162936 | up |
| 8163013 | up |
| 8163084 | down |
| 8163672 | down |
| 8165995 | up |
| 8166079 | down |
| 8166098 | up |
| 8166703 | down |
| 8166723 | down |
| 8167640 | down |
| 8168079 | up |
| 8168161 | down |
| 8168291 | up |
| 8168420 | up |
| 8168567 | down |
| 8168940 | up |
| 8169009 | down |
| 8169061 | up |
| 8169080 | up |
| 8169473 | up |
| 8170322 | up |
| 8170786 | down |
| 8171229 | up |
| 8171624 | down |
| 8171823 | up |
| 8173503 | up |
| 8177931 | up |
| 8180392 | up |

The table shows expression trends (i.e., up-regulation or down-regulation) of GRPS predictor genes in MUTZ-3 cells exposed to respiratory sensitizer. The Affymetrix Probe Set ID for the Human ST 1.0 Array are provided.

TABLE 1
Concentrations and vehicles used for each reference compound
during assay development

| | | | | | GARD input |
|---|---|---|---|---|---|
| | | | Max solubility | Rv90 | concentratrion |
| Compound | Abbreviation | Vehicle | (μM) | (μM) | (μM) |
| Respiratory sensitizers | | | | | |
| Ammonium | AH | Water | 35 | — | 35 |
| hexachloroplatinate | | | | | |
| Ammonium persulfate | AP | DMSO | — | — | 500 |
| Ethylenediamine | EDA | Water | — | — | 500 |
| Glutaraldehyde | GA | Water | — | 10 | 10 |
| Hexamethylen diisocyanate | HDI | DMSO | 100 | — | 100 |
| Maleic Anhydride | MA | DMSO | — | — | 500 |
| Methylene diphenol | MDI | DMSO | 50 | — | 50 |
| diisocyanate | | | | | |
| Phtalic Anhydride | PA | DMSO | 200 | — | 200 |
| Toluendiisocyanate | TDI | DMSO | 40 | — | 40 |
| Trimellitic anhydride | TMA | DMSO | 150 | — | 150 |
| Non-Respiratory sensitizers | | | | | |
| 1-Butanol | BUT | DMSO | — | — | 500 |
| 2-Aminophenol | 2AP | DMSO | — | 100 | 100 |
| 2-Hydroxyethyl acrylate | 2HA | Water | — | 100 | 100 |
| 2-nitro-1,4-Phenylenediamine | NPDA | DMSO | — | 300 | 300 |
| 4-Aminobenzoic acid | PABA | DMSO | — | — | 500 |
| Chlorobenzene | CB | DMSO | 98 | — | 98 |
| Dimethyl formamide | DF | Water | — | — | 500 |
| Ethyl vanillin | EV | DMSO | — | — | 500 |
| Formaldehyde | FA | Water | — | 80 | 80 |
| Geraniol | GER | DMSO | — | — | 500 |
| Hexylcinnamic aldehyde | HCA | DMSO | 32.34 | — | 32.34 |
| Isopropanol | IP | Water | — | — | 500 |
| Kathon CG* | KCG | Water | — | 0.0035% | 0.0035% |
| Methyl salicylate | MS | DMSO | — | — | 500 |
| Penicillin G | PEN G | Water | — | — | 500 |
| Propylene glycol | PG | Water | — | — | 500 |
| Potassium Dichromate | PD | Water | 51.02 | 1.5 | 1.5 |
| Potassium permanganate | PP | Water | 38 | — | 38 |
| Tween 80 | T80 | DMSO | — | — | 500 |
| Zinc sulphate | ZS | Water | 126 | — | 126 |

*The chemical Kathon CG is a mixture of the two compounds MC and MCI. The concentration of the mixture is given in %.

TABLE 2
Chemicals included in the independent dataset used for validation of GRPS

| | | | | | GARD input |
|---|---|---|---|---|---|
| | | | Max solubility | Rv90 | concentratrion |
| Compound | Abbreviation | Vehicle | (μM) | (μM) | (μM) |
| Respiratory sensitizers | | | | | |
| Chloramine T | CH-T | Water | — | — | 500 |
| Ethylenediamine | EDA | Water | — | — | 500 |
| Isophorone diisocyanate | IPDI | DMSO | 25 | — | 25 |
| Phtalic Anhydride | PA | DMSO | 200 | — | 200 |
| Piperazine | PPZ | Water | — | — | 500 |
| Reactive Orange | RO | Water | — | 100 | 100 |
| Non-respiratory sensitizers | | | | | |
| 1-Butanol | BUT | DMSO | — | — | 500 |
| 2,4-dinitrochlorobenzene | DNCB | DMSO | — | 4 | 4 |
| 2-mercaptobenzothiazole | MBT | DMSO | 250 | — | 250 |
| Benzaldehyde | BA | DMSO | 250 | — | 250 |
| Chlorobenzene | CB | DMSO | 98 | — | 98 |
| Cinnamyl alcohol | CALC | DMSO | 500 | — | 500 |
| Diethyl phthalate | DP | DMSO | 50 | — | 50 |
| Eugenol | EU | DMSO | 649 | 300 | 300 |
| Glycerol | GLY | Water | — | — | 500 |
| Glyoxal | GO | Water | — | 300 | 300 |
| Isoeugenol | IEU | DMSO | 641 | 300 | 300 |
| Lactic acid | LA | Water | — | — | 500 |
| Octanoic acid | OA | DMSO | 504 | — | 500 |
| Phenol | PHE | Water | — | — | 500 |
| p-hydroxybenzoic acid | HBA | DMSO | 250 | — | 250 |
| p-phenylenediamine | PPD | DMSO | 566 | 75 | 75 |
| Resorcinol | RC | Water | — | — | 500 |
| Salicylic acid | SA | DMSO | — | — | 500 |
| Sodium dodecyl sulphate | SDS | Water | — | 200 | 200 |

TABLE 3
Results from SVM classifications of the independent test dataset

| | | | | | | Classification¹ |
|---|---|---|---|---|---|---|
| | SVM decision value | | | | | Pos if 1 |
| Treatment | 1 | 2 | 3 | 4 | 5 | sample >0 |

Respiratory sensitizers

| | | | |
|---|---|---|---|
| Chloramine T | 0.52 | 0.59 | Sensitizer |
| Ethylenediamine | −0.32 | −0.20 | Non-sensitizer |
| Isophorone | 0.10 | 0.17 | Sensitizer |
| diisocyanate | | | |
| Phtalic Anhydride | 0.20 | −0.12 | Sensitizer |
| Piperazine | −0.05 | −0.12 | Non-sensitizer |
| Reactive Orange | 0.41 | 0.41 | sensitizer |

Non-respiratory sensitizers

| | | | | | | |
|---|---|---|---|---|---|---|
| 1-Butanol | −0.32 | 0.12 | | | | Sensitizer |
| 2,4-dinitrochloro- | −1.66 | −1.18 | −1.90 | | | Non-sensitizer |
| benzene | | | | | | |
| 2-mercaptobenzo- | −0.44 | −0.43 | −0.57 | | | Non-sensitizer |
| thiazole | | | | | | |
| Benzaldehyde | −0.79 | −0.87 | −0.70 | | | Non-sensitizer |
| Chlorobenzene | −1.03 | −0.76 | −1.15 | 0.24 | 0.06 | Sensitizer |
| Cinnamyl alcohol | −0.57 | −1.44 | −1.26 | | | Non-sensitizer |
| Diethyl phthalate | −1.37 | −0.96 | −1.22 | | | Non-sensitizer |
| Eugenol | −1.67 | −1.53 | −1.51 | | | Non-sensitizer |
| Glycerol | −1.05 | −1.11 | −0.77 | | | Non-sensitizer |
| Glyoxal | −1.02 | −0.69 | −0.56 | | | Non-sensitizer |
| Isoeugenol | −1.44 | −1.27 | −1.32 | | | Non-sensitizer |
| Lactic acid | −1.20 | −0.81 | −0.89 | | | Non-sensitizer |
| Octanoic acid | −0.65 | −0.79 | −1.22 | | | Non-sensitizer |
| Phenol | −1.04 | −0.38 | −0.95 | | | Non-sensitizer |
| p-hydroxybenzoic | −0.81 | −0.56 | −1.09 | | | Non-sensitizer |
| acid | | | | | | |
| p-phenylenediamine | −1.38 | −1.19 | −1.80 | | | Non-sensitizer |
| Resorcinol | −1.01 | −0.99 | −1.40 | | | Non-sensitizer |
| Salicylic acid | −0.73 | −1.08 | −1.13 | | | Non-sensitizer |
| Sodium dodecyl | −1.49 | −0.80 | −1.30 | | | Non-sensitizer |
| sulphate | | | | | | |

¹Classification on sensitizing properties for each chemical compound was based on the rule stating that any given sample in the test dataset should be classified as a respiratory sensitizer if any of replicate stimulations have a SVM decision value > 0.

TABLE 4
Canonical pathways associated with the top 999 predictors able to
separate respiratory chemical sensitizers from non-respiratory sensitizers

| | −log(p- | |
|---|---|---|
| Canonical Pathway | value) | Regulated molecules¹ |
| Oxidative phosphorylation | 17.62 | ATP5E, ATP5I, ATPK, COX Vb, COX VIIa-2, NDUFA1, NDUFA13, |
| | | NDUFA2, NDUFA3, NDUFA6, NDUFA7, NDUFA9, NDUFAB1, |
| | | NDUFB10, NDUFB4, NDUFB6, NDUFB8, NDUFB9, |
| | | NDUFC1, NDUFS4, NDUFS5, NDUFS6, NDUFS8, NDUFV2, |
| | | UQCR10, UQCRQPC |
| Ubiquinone metabolism | 13.29 | NDUFA1, NDUFA13, NDUFA2, NDUFA3, NDUFA6, NDUFA7, |
| | | NDUFA9, NDUFAB1, NDUFB10, NDUFB4, NDUFB6, NDUFB8, |
| | | NDUFB9, NDUFC1, NDUFS4, NDUFS5, NDUFS6, |
| | | NDUFS8, NDUFV2 |
| Granzyme B signaling | 4.72 | Bid, Caspase-2, Lamin A/C, LAMP2, Smac/Diablo, tBid, Tubulin |
| | | alpha |
| FAS signaling cascades | 3.79 | Bid, c-FLIP (S), Caspase-2, DAXX, Lamin A/C, Smac/Diablo, tBid |
| Cytoplasmic/mitochondrial transport of proapoptotic | 3.57 | Bid, DAXX, DLC1 (Dynein LC8a), DLC2 (Dynein LC8b), |
| proteins Bid, Bmf and Bim | | Smac/Diablo, tBid |
| Inhibitory PD-1 signaling in T cells | 3.27 | BCL2L1, CD8, CD8 alpha, CD80, CD86, MHC class II, PTEN |
| HSP60 and HSP70/TLR signaling pathway | 3.22 | CD80, CD86, MD-2, MEK1/2, MHC class II, MyD88, Ubiquitin |
| Astrocyte differentiation from adult stem cells | 3.17 | HES1, ID1, ID2, ID3, MEK1/2, SOX1 |
| Apoptotic TNF-family pathways | 3.06 | Apo-2L(TNFSF10), BCL2L1, Bid, Caspase-2, Smac/Diablo, tBid |
| TNFR1 signaling pathway | 3.00 | Bid, c-FLIP (S), Caspase-2, jBid, Smac/Diablo, tBid |
| Role of Nek in cell cycle regulation | 2.80 | Histone H1, Ran, Tubulin (in microtubules), Tubulin alpha, Tubulin |
| | | beta |
| ATP/ITP metabolism | 2.70 | 5′-NTC, ADSL, APRT, POLR2G, POLR2J, PPAP, RPB10, RPB6, |
| | | RPB8, RRP41 |
| Generation of memory CD4+ T cells | 2.51 | BCL2L1, CD80, CD86, IL7RA, MHC class II |
| Dynein-dynactin motor complex in axonal transport | 2.48 | DYNLL, DYNLT, Tctex-1, TMEM108, Tubulin (in microtubules), |
| in neurons | | Ubiquitin |
| Antigen presentation by MHC class II | 2.44 | HLA-DM, HLA-DRA1, MHC class II |
| IL-33 signaling pathway | 2.36 | Histone H2A, Histone H2B, MEK1/2, MyD88, ST2L, Ubiquitin |
| Insulin regulation of translation | 2.27 | eEF2, eIF4A, eIF4B, eIF4G1/3, MEK1(MAP2K1) |
| TNF-alpha-induced Caspase-8 signaling | 2.23 | Bid, c-FLIP (S), Caspase-2, PP2A regulatory, tBid |
| Antigen presentation by MHC class I | 2.18 | CD8, CD8 alpha, PSMB5, PSME3 |
| Main pathways of Schwann cells transformation in | 2.18 | BCL2L1, Calmodulin, MEK1(MAP2K1), MEK1/2, Neuregulin 1, PTEN |
| neurofibromatosis type 1 | | |
| Granzyme A signaling | 2.08 | Histone H1, Histone H2B, Lamin A/C, LAMP2 |
| G-CSF-induced myeloid differentiation | 2.08 | G-CSF receptor, MEK1/2, Myeloblastin, PERM |
| Substance P mediated membrane blebbing | 2.07 | MRLC, Tubulin (in microtubules), Tubulin alpha |
| Role of IAP-proteins in apoptosis | 2.03 | Bid, Smac/Diablo, tBid, Ubiquitin |

¹Molecules indicated in bold are present in GRPS.

1. A method for identifying agents capable of inducing respiratory sensitization in a mammal comprising or consisting of the steps of:
a) exposing a population of dendritic cells or a population of dendritic-like cells to a test agent; and
b) measuring in the cells the expression of one or more biomarker(s) selected from the group defined in Table A(i) and/or Table A(ii);
wherein the expression in the cells of the one or more biomarkers measured in step (b) is indicative of the respiratory sensitizing effect of the test agent.

2. The method according to claim 1 further comprising:
c) exposing a separate population of the dendritic cells or dendritic-like cells to one or more negative control agent that is not a respiratory sensitizer in a mammal; and
d) measuring in the cells the expression of the one or more biomarker(s) measured in step (b)
wherein the test agent is identified as a respiratory sensitizer in the event that the presence and/or amount in the test sample of the one or more biomarker measured in step (d) differs from the presence and/or amount in the control sample of the one or more biomarker measured in step (b).

3. The method according to claim 1 or 2 further comprising:
e) exposing a separate population of the dendritic cells or dendritic-like cells to one or more positive control agent that is a respiratory sensitizer in a mammal; and
f) measuring in the cells the expression of the one or more biomarker(s) measured in step (b)
wherein the test agent is identified as a respiratory sensitizer in the event that the presence and/or amount in the test sample of the one or more biomarker measured in step (f) corresponds to the presence and/or amount in the positive control sample of the one or more biomarker measured in step (b).

4. The method according to any one of the preceding claims wherein step (b) comprises or consists of measuring the expression of one or more biomarkers defined in Table A(ii) for example, at least 2 or 3 of the biomarkers defined in Table 1A.

5. The method according to any one of the preceding claims wherein step (b) comprises or consists of measuring the expression of TNFRSF19.

6. The method according to any one of the preceding claims wherein step (b) comprises or consists of measuring the expression of SNORA74A.

7. The method according to any one of the preceding claims wherein step (b) comprises or consists of measuring the expression of SPAM1.

8. The method according to any one of the preceding claims wherein step (b) comprises or consists of measuring the expression of TNFRSF19, SNORA74A and SPAM1.

9. The method according to any one of the preceding claims wherein step (b) comprises or consists of measuring in step (b) the expression of one or more biomarkers defined in Table A(ii), for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341 or 342 of the biomarkers defined in Table A(ii).

10. The method according to any one of the preceding claims wherein step (b) comprises or consists of measuring the expression of all of the biomarkers defined in Table A(ii).

11. The method according to any one of the preceding claims wherein step (b) comprises or consists of measuring the expression of one or more of the biomarkers defined in Table A(iii), for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43 or 44 of the biomarkers defined in Table A(iii).

12. The method according to any one of the preceding claims wherein step (b) comprises or consists of measuring the expression of all of the biomarkers defined in Table A(iii).

13. The method according to any one of the preceding claims wherein step (b) comprises or consists of measuring the expression of all of the biomarkers defined in Table A.

14. The method according to any one of the preceding claims wherein step (b) comprises measuring the expression of a nucleic acid molecule encoding the one or more biomarker(s).

15. The method according to claim 14 wherein the nucleic acid molecule is a cDNA molecule or an mRNA molecule.

16. The method according to claim 14 wherein the nucleic acid molecule is an mRNA molecule.

17. The method according to claim 14 wherein the nucleic acid molecule is a cDNA molecule.

18. The method according to any one of claims 14 to 17 wherein measuring the expression of the one or more biomarker(s) in step (b) is performed using a method selected from the group consisting of Southern hybridisation, Northern hybridisation, polymerase chain reaction (PCR), reverse transcriptase PCR (RT-PCR), quantitative real-time PCR (qRT-PCR), nanoarray, microarray, macroarray, autoradiography and in situ hybridisation.

19. The method according to any one of claims 14 to 18 wherein measuring the expression of the one or more biomarker(s) in step (b) is determined using a DNA microarray.

20. The method according to any one of the preceding claims wherein measuring the expression of the one or more biomarker(s) in step (b) is performed using one or more binding moieties, each capable of binding selectively to a nucleic acid molecule encoding one of the biomarkers identified in Table A.

21. The method according to claim 20 wherein the one or more binding moieties each comprise or consist of a nucleic acid molecule.

22. The method according to claim 21 wherein the one or more binding moieties each comprise or consist of DNA, RNA, PNA, LNA, GNA, TNA or PMO.

23. The method according to claim 20 or 22 wherein the one or more binding moieties each comprise or consist of DNA.

24. The method according to any one of claims 21 to 24 wherein the one or more binding moieties are 5 to 100 nucleotides in length.

25. The method according to any one of claims 21 to 25 wherein the one or more nucleic acid molecules are 15 to 35 nucleotides in length.

26. The method according to any one of claims 21 to 26 wherein the binding moiety comprises a detectable moiety.

27. The method according to claim 26 wherein the detectable moiety is selected from the group consisting of: a fluorescent moiety; a luminescent moiety; a chemiluminescent moiety; a radioactive moiety (for example, a radioactive atom); or an enzymatic moiety.

28. The method according to claim 26 wherein the detectable moiety comprises or consists of a radioactive atom.

29. The method according to claim 28 wherein the radioactive atom is selected from the group consisting of technetium-99m, iodine-123, iodine-125, iodine-131, indium-111, fluorine-19, carbon-13, nitrogen-15, oxygen-17, phosphorus-32, sulphur-35, deuterium, tritium, rhenium-186, rhenium-188 and yttrium-90.

30. The method according to claim 27 wherein the detectable moiety of the binding moiety is a fluorescent moiety.

31. The method according to any one of claims 1 to 22 wherein step (b) comprises or consists of measuring the expression of the protein of the one or more biomarker defined in step (b).

32. The method according to claim 31 wherein measuring the expression of the one or more biomarker(s) in step (b) is performed using one or more binding moieties each capable of binding selectively to one of the biomarkers identified in Table A.

33. The method according to claim 32 wherein the one or more binding moieties comprise or consist of an antibody or an antigen-binding fragment thereof.

34. The method according to claim 33 wherein the antibody or fragment thereof is a monoclonal antibody or fragment thereof.

35. The method according to claim 33 or 34 wherein the antibody or antigen-binding fragment is selected from the group consisting of intact antibodies, Fv fragments (e.g. single chain Fv and disulphide-bonded Fv), Fab-like fragments (e.g. Fab fragments, Fab′ fragments and F(ab)2 fragments), single variable domains (e.g. V_{H} and V_{L} domains) and domain antibodies (dAbs, including single and dual formats [i.e. dAb-linker-dAb]).

36. The method according to claim 35 wherein the antibody or antigen-binding fragment is a single chain Fv (scFv).

37. The method according to claim 32 wherein the one or more binding moieties comprise or consist of an antibody-like binding agent, for example an affibody or aptamer.

38. The method according to any one of claims 32 to 37 wherein the one or more binding moieties comprise a detectable moiety.

39. The method according to claim 38 wherein the detectable moiety is selected from the group consisting of a fluorescent moiety, a luminescent moiety, a chemiluminescent moiety, a radioactive moiety and an enzymatic moiety.

40. The method according to any one of the preceding claims wherein step (b) is performed using an array.

41. The method according to claim 40 wherein the array is a bead-based array.

42. The method according to claim 41 wherein the array is a surface-based array.

43. The method according to any one of claims 40 to 42 wherein the array is selected from the group consisting of: macroarray; microarray; nanoarray.

44. An array for use in a method according any one of the preceding claims, the array comprising one or more first binding agents as defined in any one of claims 20 to 30 and 32 to 39.

45. An array according to claim 44 comprising binding agents which are collectively capable of binding to all of the biomarkers defined in Table 1.

46. An array according to claim 44 or 45 wherein the first binding agents are immobilised.

47. The method according to any one of the preceding claims for identifying agents capable of inducing a respiratory hypersensitivity response.

48. The method according to any one of the preceding claims wherein the hypersensitivity response is a humoral hypersensitivity response.

49. The method according to claim 47 or 48 wherein the hypersensitivity response is a type I hypersensitivity response.

50. The method according to any one of the preceding claims for identifying agents capable of inducing respiratory allergy.

51. The method according to any one of the preceding claims wherein the population of dendritic cells or population of dendritic-like cells is a population of dendritic-like cells.

52. The method according to claim 51 wherein the dendritic-like cells are myeloid dendritic-like cells.

53. The method according to claim 52 wherein the myeloid dendritic-like cells are derived from myeloid dendritic cells.

54. The method according to claim 53 wherein the cells derived from myeloid dendritic cells are myeloid leukaemia-derived cells.

55. The method according to claim 54 wherein the myeloid leukaemia-derived cells are selected from the group consisting of KG-1, THP-1, U-937, HL-60, Monomac-6, AML-193 and MUTZ-3.

56. The method according to any one of the preceding claims wherein the dendritic-like cells are MUTZ-3 cells.

57. The method according to any one of the claims 2 to 56 wherein the one or more negative control agent provided in step (c) is selected from the group consisting of 1-Butanol; 2-Aminophenol; 2-Hydroxyethyl acrylate; 2-nitro-1,4-Phenylenediamine; 4-Aminobenzoic acid; Chlorobenzene; Dimethyl formamide; Ethyl vanillin; Formaldehyde; Geraniol; Hexylcinnamic aldehyde; Isopropanol; Kathon CG*; Methyl salicylate; Penicillin G; Propylene glycol; Potassium Dichromate; Potassium permanganate; Tween 80; and Zinc sulphate.

58. The method according to claim 57 wherein at least 2 control non-sensitizing agents are provided, for example, at least 3, 4, 5, 6, 7, 8, 9, 10, 11, 12, 13, 14, 15, 16, 17, 18, 19 or at least 20 control non-sensitizing agents.

59. The method according to any one of claims 3 to 58 wherein the one or more positive control agent provided in step (e) comprises or consists of one or more agent selected from the group consisting of ammonium hexachloroplatinate, ammonium persulfate, glutaraldehyde, hexamethylen diisocyanate, maleic anhydride, methylene diphenol diisocyanate, phtalic anhydride, toluendiisocyanate and trimellitic anhydride.

60. The method according to claim 59 wherein at least 2 control sensitizing agents are provided, for example, at least 3, 4, 5, 6, 7, 8, 9 or at least 10 control sensitizing agents.

61. The method according to any one of the preceding claims wherein the method is indicative of the sensitizing potency of the sample to be tested.

62. An array for use in a method according any one of the preceding claims, the array comprising one or more binding moieties as defined in any one of claims 20 to 30 and 32 to 39.

63. An array according to claim 62 wherein the binding moieties are capable of binding to all of the biomarkers defined in Table A(i).

64. An array according to claim 62 or 63 wherein the binding moieties are capable of binding to all of the biomarkers defined in Table A(ii).

65. An array according to claim 62, 63 or 64 wherein the binding moieties are capable of binding to all of the biomarkers defined in Table A(iii).

66. An array according to any one of claims 62 to 65 wherein the binding moieties are capable of binding to all of the biomarkers defined in Table A.

67. An array according to any on of claims 62 to 65 wherein the binding moieties are immobilised.

68. Use of two or more biomarkers selected from the group defined in Table A in combination for identifying respiratory hypersensitivity response sensitising agents.

69. The use according to claim 68 wherein all of the biomarkers defined in Table A are used collectively for identifying hypersensitivity response sensitising agents.

70. Use of one or more binding moiety as defined in any one of claims 20 to 30 or 32 to 39 for identifying respiratory hypersensitivity response sensitising agents.

71. The use according to claim 70 wherein all of the biomarkers defined in Table A are used collectively for identifying hypersensitivity response sensitising agents

72. An analytical kit for use in a method according any one of claims 1 to 61 comprising:
A) an array according to any one of claims 62 to 67 and/or one or more binding moiety as defined in any one of claims 20 to 30 or 32 to 39; and
B) instructions for performing the method as defined in any one of claims 1 to 60 (optional).

73. An analytical kit according to claim 72 further comprising one or more control samples.

74. An analytical kit according to claim 73 comprising one or more non-sensitizing agent(s).

75. An analytical kit according to claim 72, 73 or 74 comprising one or more sensitizing agent(s).

76. A method of treating or preventing a respiratory type I hypersensitivity reaction (such as respiratory asthma) in a patient comprising the steps of:
(a) providing one or more test agent that the patient is or has been exposed to;
(b) determining whether the one or more test agent provided in step (a) is a respiratory sensitizer using a method provided in the first aspect of the present invention; and
(c) where one or more test agent is identified as a respiratory sensitizer, reducing or preventing exposure of the patient to the one or more test agent identified as a respiratory sensitizer and/or providing appropriate treatment for the symptoms of sensitization.

77. The method according to claim 76 wherein the treatment of the symptoms of sensitization is selected from the group consisting of short-acting beta2-adrenoceptor agonists (SABA), such as salbutamol; anticholinergic medications, such as ipratropium bromide; other adrenergic agonists, such as inhaled epinephrine; Corticosteroids such as beclomethasone; long-acting beta-adrenoceptor agonists (LABA) such as salmeterol and formoterol;
leukotriene antagonists such as montelukast and zafirlukast; and/or mast cell stabilizers (such as cromolyn sodium).

78. A computer program for operating the method defined in the first aspect of the invention.

79. The computer program according to claim 78 wherein the computer program is recorded on a computer-readable carrier.

80. A method or use substantially as described herein.

81. An array, kit or computer program substantially as described herein.
